SEQUENCE LISTING (12 sequences)

SEQ ID NO: 1 - protein, 31 aa, Artificial Sequence
  Synthetic peptide
  LIPID (1)..(1): N-terminus modified with myristoyl group
GWTLNSAGYLLGKINLKALAALAKKILGGCC

SEQ ID NO: 2 - protein, 33 aa, Artificial sequence
  Synthetic peptide
  MOD_RES (1)..(1): N-terminus modified with 5-carboxyfluorescein
CCGGYTIWMPENPRPGTPCDIFTNSRGKRASNG

SEQ ID NO: 3 - DNA, 21 bp, Artificial Sequence
  Synthetic oligonucleotide
  misc_feature (20)..(21): deoxyribonucleotides
caaguuccaucgugucauctt

SEQ ID NO: 4 - DNA, 21 bp, Artificial Sequence
  Synthetic oligonucleotide
  misc_feature (20)..(21): deoxyribonucleotides
gaugacacgauggaacuugtt

SEQ ID NO: 5 - DNA, 21 bp, Artificial Sequence
  Synthetic oligonucleotide
  misc_feature (20)..(21): deoxyribonucleotides
gaaagagcaucuauggugatt

SEQ ID NO: 6 - DNA, 21 bp, Artificial Sequence
  Synthetic oligonucleotide
  misc_feature (20)..(21): deoxyribonucleotides
ucaccauagaugcucuuuctt

SEQ ID NO: 7 - DNA, 21 bp, Artificial Sequence
  Synthetic oligonucleotide
  misc_feature (20)..(21): deoxyribonucleotides
cuuacgcugaguacuucgatt

SEQ ID NO: 8 - DNA, 21 bp, Artificial Sequence
  Synthetic oligonucleotide
  misc_feature (20)..(21): deoxyribonucleotides
ucgaaguacucagcguaagtt

SEQ ID NO: 9 - DNA, 24 bp, Artificial Sequence
  Synthetic oligonucleotide
ggaaagactgttccaaaaacagtg

SEQ ID NO: 10 - DNA, 23 bp, Artificial Sequence
  Synthetic oligonucleotide
gtcttggtgctctccaccttccg

SEQ ID NO: 11 - DNA, 20 bp, Artificial Sequence
  Synthetic oligonucleotide
gtcaacgggggacataaaag

SEQ ID NO: 12 - DNA, 23 bp, Artificial Sequence
  Synthetic oligonucleotide
caacaatcaagacattctttcca

(19) United States
(12) Patent Application Publication
(10) Pub. No.: US 2021/0177770 A1
(43) Pub. Date: Jun. 17, 2021

(54) POROUS SILICON MATERIALS COMPRISING A METAL SILICATE FOR DELIVERY OF THERAPEUTIC AGENTS

(71) Applicants: SPINNAKER BIOSCIENCES, INC., Solana Beach, CA (US); THE REGENTS OF THE UNIVERSITY OF CALIFORNIA, Oakland, CA (US); MASSACHUSETTS INSTITUTE OF TECHNOLOGY, Cambridge, MA (US)

(72) Inventors: Michael J. SAILOR, La Jolla, CA (US); Jinyoung KANG, La Jolla, CA (US); Jinmyoung JOO, Seoul (KR); Emily ANGLIN, San Marcos, CA (US); Ester KWON, Cambridge, MA (US); Matthew SKALAK, Boston, MA (US); Sangeeta BHATIA, Lexington, MA (US)

(73) Assignees: SPINNAKER BIOSCIENCES, INC., Solana Beach, CA (US); THE REGENTS OF THE UNIVERSITY OF CALIFORNIA, Oakland, CA (US); MASSACHUSETTS INSTITUTE OF TECHNOLOGY, Cambridge, MA (US)

(21) Appl. No.: 16/093,131

(22) PCT Filed: Apr. 14, 2017
(86) PCT No.: PCT/US17/27772

§ 371 Date: Oct. 11, 2018

Related U.S. Application Data
(60) Provisional application No. 62/322,782, filed on Apr. 14, 2016.

(51) Int. Cl. A61K 9/51; A61K 49/00; A61K 38/16; A61K 38/17; C12N 15/113

(52) U.S. Cl. CPC A61K 9/5115; A61K 49/0013; A61K 9/5192; A61K 38/162; C12N 2310/14; A61K 49/0004; A61K 49/0093; C12N 15/113; A61K 38/17

(57) ABSTRACT

Compositions useful in the controlled delivery of therapeutic agents and their methods of preparation and use are provided. The compositions comprise an optionally oxidized porous silicon core, a layer on the surface of the porous silicon core that comprises a metal silicate, and a therapeutic agent. The compositions optionally further comprise one or more targeting agents and/or cell-penetrating agents to enable the particles to target and enter cells or tissues of interest in a treated subject.

### CROSS-REFERENCE TO RELATED APPLICATION

This application claims the benefit of U.S. Provisional Application No. 62/322,782, filed on Apr. 14, 2016, the disclosure of which is incorporated herein by reference in its entirety.

### GOVERNMENT SUPPORT

This invention was made with government support under contract number R24EY022025-01 and grant number NRSA 1F32CA177094-01 awarded by the National Institutes of Health, grant number DMR1210417 awarded by the National Science Foundation, and cooperative agreement number HR0011-13-2-0017 awarded by the Defense Advanced Research Projects Agency. The government has certain rights in the invention.

### BACKGROUND OF THE INVENTION

There is considerable interest in developing drug delivery systems that provide sustained and reliable release of therapeutic agents. Such systems can be designed to deliver the therapeutic agent to any tissue of a subject in need of treatment. Depending on the target tissue, the drug delivery vehicle may be administered by oral, transmucosal, topical, injection, or inhalation routes. Release of the agent from the drug delivery vehicle within the tissue should be sufficiently rapid that a therapeutically effective concentration of the agent is achieved within the target tissue, while at the same time, release should not be so high that the agent either reaches toxic levels in the tissue or is wasted by degradative metabolism.

In the case of unstable therapeutic agents, sustained and reliable delivery is all the more difficult due to stability issues. In addition, targeted delivery of therapeutic agents to specific tissues may be desirable to increase the effectiveness of treatment at the affected tissue and to minimize side effects at unaffected tissues. The unique properties and environment of a given target tissue may also provide both challenges and opportunities in the design of a drug delivery system.

Exemplary drug delivery vehicles include liposomes, organic microspheres, drug-polymer conjugates, inorganic carriers, and the like. Among the inorganic carriers, inorganic nanoparticles have recently become attractive candidates for use in drug delivery systems due to their unique physicochemical properties, in particular their adaptable sizes, shapes, surface reactivity, and solubility. Examples of nanoparticles, including inorganic nanoparticles, usefully employed as drug delivery vehicles include calcium phosphate nanoparticles, carbon nanotubes, gold nanoparticles, graphene oxide nanoparticles, iron oxide nanoparticles, mesoporous silica nanoparticles, and the like.

For example, Xue et al. (2009) Acta Biomater. 5:1686 report the use of mesoporous calcium silicate for the controlled adsorption and release of protein agents. In this study, calcium silicate precipitates were formed from solution phase. The precipitates were treated with acid to generate a mesoporous structure on the surface of the particles, thus increasing the surface area of the particles, improving their bioactivity, and strengthening protein interactions with the surface.

Salinas et al. (2001) J. Sol-Gel Sci. Techn. 21:13 have generated gel glasses, including calcium silicate gel glasses, by sol-gel methods. The properties of these materials have been examined in the presence of a simulated body fluid using a dynamic assay model.

Wu et al. (2010) Adv. Mater. 22:749 have synthesized nanostructured mesoporous calcium silicate hydrate spheres from the solution phase using surfactant-free sonochemical methods. The physicochemical properties of these materials were assessed, including the capability of the materials as drug carriers.

Li et al. (2007) J. Biomed. Mater. Res. B. 83B:431 report the preparation of mesoporous amorphous calcium silicates from solution using a template route. The materials were found to display high bone-forming activity in an in vitro model compared to a conventional amorphous calcium silicate.

Wu et al. (2012) J. Mater. Chem. 22:16801 describe the use of bioactive mesoporous calcium silicate nanoparticles in filling the apex roots of teeth. The nanoparticles used in this study were synthesized by precipitation from solution using a cationic detergent template.

Kokubo et al. (2003) Biomaterials 24:2161 review the development of inorganic bioactive materials with improved mechanical properties for use as bone substitutes. Such materials include glass ceramics that may form amorphous calcium silicate intermediates on their surfaces during the deposition of apatite in the presence of simulated body fluid.

Despite the above reports, there is a continuing need to develop improved compositions, methods, and systems for the delivery of therapeutic agents, in particular for the targeted delivery of therapeutic agents to diseased tissues.

### SUMMARY OF THE INVENTION

The present disclosure addresses these and other needs by providing in one aspect compositions for delivering a therapeutic agent comprising a particle comprising a porous silicon core, a layer on the surface of the core comprising a metal silicate, and a therapeutic agent.

In some embodiments, the layer on the surface of the particle is formed by treating a porous silicon precursor particle with an aqueous solution comprising the therapeutic agent and a metal salt, and more specifically the aqueous solution comprises a concentration of metal salt of at least 0.1 molar.

In some embodiments, the layer on the surface of the particle comprises a divalent metal silicate, such as a calcium silicate.

In some embodiments, the porous silicon core has a diameter of about 1 nm to about 1 cm, and more specifically, the layer on the surface of the porous silicon core may have a thickness of between 1 and 90 percent of the diameter of the core.

In embodiments, the particle is a photoluminescent particle that may emit light in a range from 500 nm to 1000 nm.

In some embodiments, the porous silicon core comprises an etched crystalline silicon material, such as an electrochemically etched crystalline silicon material or a chemical stain etched crystalline silicon material. In some embodiments, the porous silicon core comprises a microporous etched silicon material, such as a microporous etched silicon material comprising a plurality of pores with an average pore diameter of at most about 1 nm. In other embodiments, the porous silicon core comprises a mesoporous etched silicon material, such as a mesoporous etched silicon material comprising a plurality of pores with an average pore diameter of from about 1 nm to about 50 nm. In still other embodiments, the porous silicon core comprises a macroporous etched silicon material, such as a macroporous etched silicon material comprising a plurality of pores with an average pore diameter of from about 50 nm to about 1000 nm.

In some embodiments, the therapeutic agent is a small-molecule agent, a vitamin, an imaging agent, a protein, a peptide, a nucleic acid, an oligonucleotide, an aptamer, or a mixture thereof, such as a negatively-charged therapeutic agent, for example an oligonucleotide. In some embodiments the porous silicon particle comprises a targeting agent, a cell-penetrating agent, or both a targeting agent and a cell-penetrating agent. In some embodiments, the porous silicon core comprises an oxidized porous silicon material.

In another aspect, the disclosure provides pharmaceutical compositions comprising any of the instant compositions and a pharmaceutically acceptable carrier.

In yet another aspect, the disclosure provides methods of preparing a particle for delivery of a therapeutic agent comprising the steps of:

providing a porous silicon precursor particle;

treating the porous silicon precursor particle with an aqueous solution comprising the therapeutic agent and a metal salt.

According to still other aspects are provided methods of treatment comprising administration of the compositions of the disclosure to a subject in need of treatment.

### BRIEF DESCRIPTION OF THE DRAWINGS

FIG. 1. Schematic illustration of an exemplary process for the preparation of siRNA-loaded, calcium silicate-coated porous silicon nanoparticles (Ca-pSiNP-siRNA).

FIGS. 2A-2E. Transmission electron microscope (TEM) images of pSiNP (FIG. 2A), Ca-pSiNP (FIG. 2B), and Ca-pSiNP-siRNA (FIG. 2C) formulations. Scale bar is 200 nm. FIG. 2D shows cryogenic nitrogen adsorption-desorption isotherms for pSiNP and Ca-pSiNP formulations. FIG. 2E shows photoluminescence emission spectra (λₑₓ: 365 nm) obtained during reaction of pSiNP with 3 M or 4 M aqueous CaCl₂) solution, used to prepare the Ca-pSiNP formulation. Typical of quantum confinement, as the porous silicon core becomes thinner, the emission spectrum shifts to the blue. The growth of an electronically passivating surface layer and suppression of nonradiative recombination centers is evident in the strong increase in photoluminescence intensity observed as the reaction progresses.

FIG. 3. Silencing of relative PPIB gene expression in Neuro-2a cells after treatment with siRNA against the PPIB gene (siPPIB), aminated porous Si nanoparticle (pSiNP) loaded with siPPIB (pSiNP-siPPIB), pSiNP-siPPIB construct prepared with a calcium silicate shell and containing both cell-targeting and cell-penetrating peptides on the outer shell in a dual peptide nanocomplex (Ca-pSiNP-siPPIB-DPNC), the pSiNP-siPPIB-calcium silicate shell construct containing only a cell-penetrating peptide on the outer shell (Ca-pSiNP-siPPIB-mTP), the pSiNP-siPPIB-calcium silicate shell construct containing only the cell-targeting peptide on the outer shell (Ca-pSiNP-siPPIB-RVG), and the pSi nanoparticle-calcium silicate shell construct containing a negative control siRNA sequence against luciferase, and containing both the cell-targeting and the cell-penetrating peptides on the outer shell (Ca-pSiNP-siLuc-DPNC). The “7 days” designations indicate that the nanoparticle construct was stored in ethanol at 4° C. for 7 days prior to the experiment. The cell penetrating peptide is a myristoylated transportan, and the cell targeting peptide is a domain derived from the rabies virus glycopeptide (RVG) as described in the text. Statistical analyses were performed with Student's t test (*p<0.01, **p<0.03).

FIGS. 4A and 4B. Ex vivo fluorescence images of harvested organs after intravenous injection of (1) saline as a control, (2) Ca-pSiNP-siRNA-PEG, and (3) Ca-pSiNP-siRNA-DPNC. All siRNA constructs contained covalently attached dy677 fluorophore. FIG. 4A: Fluorescence image of injured brains obtained using infra-red imaging system Pearl Trilogy (Li-Cor). Green channel in the images corresponds to 700 nm emission from dy677, and the bright field image of the brain tissues is merged with the 700 nm emission. FIG. 4B: Fluorescence image of whole major organs taken with IVIS (xenogen) imaging system in the Cy5.5 channel (λ_{ex/em}: 675/694 nm).

FIGS. 5A and 5B. Scanning electron microscope images and elemental (EDX) data for pSiNP (FIG. 5A) and Ca-pSiNP (FIG. 5B).

FIG. 6A. Powder X-ray diffraction spectrum of pSiNP (lower dashed line) and Ca-pSiNP (upper solid line), as indicated. Peaks in the diffraction pattern of the Si nanoparticles are labeled with Miller indices, h k l, indicating the set of crystalline Si lattice planes responsible for that diffraction peak. FIG. 6B. Raman spectrum of pSiNP (lower dashed line) and Ca-pSiNP (upper solid line). FIG. 6C. Diffuse reflectance FTIR spectrum of pSiNP (lower dashed line) and Ca-pSiNP (upper solid line). Spectra are offset along the y-axis for clarity.

FIG. 7A. UV-Vis absorbance intensity (λ=405 nm) of pSiNP measured as a function of time in pH 9 buffer (triangles, dashed line) and pH 9 solution that is 3 M or 4 M in CaCl₂ (circles, solid line). The loss of absorbance is attributed to degradation of the elemental Si core in the nanoparticle; silicon absorbs 405 nm light strongly, whereas SiO₂ or silicate ions are transparent at this wavelength. FIG. 7B. Cumulative percent by mass of siRNA released as a function of time at 37° C. in PBS buffer. The pSiNP-NH₂-siRNA formulation was prepared by first grafting of amine to the pore walls of pSiNP using 2-aminopropyldimethylethyoxysilane (APDMES) and then loading siRNA via solution exposure for 2 hrs.

FIG. 8. Integrated photoluminescence intensity as a function of optical absorbance (365 nm), used to calculate quantum yield of Ca-pSiNP formulation relative to Rhodamine 6G standard. Integrated photoluminescence represents photoluminescence intensity-wavelength curve integrated between 500-980 nm. Photoluminescence intensity was measured using a QE-Pro (Ocean Optics) spectrometer, with excitation λₑₓ=365 nm and using a 460 nm long-pass emission filter.

FIG. 9. Cytotoxicity of Ca-pSiNP construct, quantified by the Calcein AM live/dead assay. Neuro2a cells were incubated with Ca-pSiNPs in triplicate in a 96-well plate. After 48 hrs, each well was treated with the assay solution, and viability was quantified by measured fluorescence intensity relative to standards.

FIG. 10. Schematic depicting the procedure for PEG modification and conjugation of dual peptides to Ca-pSiNP-siRNA. The coupling agent 2-aminopropyldimethylethoxysilane (APDMES) was grafted to the (calcium silicate and silica) surface of the nanoparticle, generating pendant primary amine groups (Ca-pSiNP-siRNA-NH₂). A functional polyethyleneglycol (PEG) linker was then coupled to the primary amines on the Ca-pSiNP-siRNA-NH₂ nanoparticle, using a maleimide-poly(ethylene-glycol)-succinimidyl carboxy methyl ester (MAL-PEG-SCM) species. The succinimidyl carboxymethyl ester forms an amide bond with primary amines. The distal end of the PEG chain contained a second functional group, maleimide. Maleimide forms covalent bonds to thiols of cysteine, allowing attachment of the neuronal targeting peptide (rabies virus glycoprotein) and cell penetrating peptide (myristoylated transportan).

FIG. 11A. Zeta potential of nanoparticles (pSiNP, Ca-pSiNP, Ca-pSiNP-NH₂, Ca-pSiNP-siPPIB, and Ca-pSiNP-siPPIB-NH₂, as described in the text), dispersed in ethanol. FIG. 11B. Size distribution of pSiNP and Ca-pSiNP-siPPIB-DPNC measured by dynamic light scattering (DLS).

FIG. 12. ATR-FTIR spectra of nanoparticle formulations (bottom to top) Ca-pSiNP-PEG, Ca-pSiNP-mTP, Ca-pSiNP-RVG, and Ca-pSiNP-DPNC, and peptides (mTP and FAM-RVG). Abbreviations of formulations as described in the text. Spectra are offset along the y-axis for clarity.

FIGS. 13A and 13B. Confocal microscope images of Neuro2a cells treated with (A) Ca-pSiNP-siPPIB-DPNC and (B) Ca-pSiNP-siPPIB-RVG for 2 hrs at 37° C. The signal from intrinsic luminescence of the silicon nanoparticle (red color in original) is observed on the surface of cells treated with Ca-pSiNP-siPPIB-RVG (FIG. 13B) and intracellularly in cells treated with Ca-pSiNP-siPPIB-DPNC (FIG. 13A), the DAPI nuclear stain (blue color in original) is observed in the cell nuclei of both images, the signal from the FAM tag on the RVG domain (green color in original) is more prevalent on the surface of cells treated with Ca-pSiNP-siPPIB-RVG (FIG. 13B), and the overlap of the silicon and FAM-RVG signals (yellow color in original due to the combination of red and green) is more prevalent intracellularly in cells treated with Ca-pSiNP-siPPIB-DPNC (FIG. 13A). Scale bar is 20 μm.

FIGS. 14A-14D. FACS analysis of Neuro2a cells treated with no particles as a control (FIG. 14A), Ca-pSiNP-siPPIB-RVG (FIG. 14B), Ca-pSiNP-siPPIB-DPNC (FIG. 14C), and Cy3-tagged siRNA-loaded Ca-pSiNP-siPPIB-DPNC (FIG. 14D). The percentages shown below the plots represent quantified proportions of cells transfected with FAM-RVG, Cy3-tagged siRNA, or overlapping of FAM-RVG and Cy3-tagged siRNA. Statistical analyses were performed with Student's t test (*p<0.04)

FIG. 15. Exemplary experimental procedure for targeted delivery of siRNA to the injured brain in vivo. 6 hrs post-injury, Ca-pSiNP-siRNA-PEG or Ca-pSiNP-siRNA-DPNC were injected. The siRNA in each formulation was labeled with dy677 fluorescent tag. After 1 hr of circulation, the mice were sacrified, perfused, and the organs harvested and imaged.

FIG. 16. X-ray diffraction spectra of freshly etched porous silicon microparticles (pSiMPs) ultrasonicated for 24 hours in either 4 M calcium chloride, 4 M magnesium chloride, or pH 9 buffer.

FIGS. 17A-17C. (A) Loading efficiency of rhodamine B (RhB) and ruthenium bipyridine (Ru(bpy)) using pH 9 buffer, 4 M CaCl₂, and 4 M MgCl₂ solutions. Release profile of (B) rhodamine B and (C) Ru(bpy) from pSiMPs after loading in pH 9 buffer, CaCl₂, and MgCl₂ solutions.

FIGS. 18A-18B. Loading capacity, drug release profile, and photoluminescence decrease profile of Ca-pSiNP loaded with (A) chloramphenicol or (B) vancomycin.

### DETAILED DESCRIPTION OF THE INVENTION

### Compositions Comprising Porous Silicon Particles

The instant disclosure provides in one aspect compositions useful in the delivery of therapeutic agents. Such compositions are particularly useful in the treatment of diseases or other conditions where the controlled release of a therapeutic agent is desired. For example, many diseases or conditions are advantageously treated by the steady release of an active therapeutic agent over a long period of time. Such treatments provide a more constant concentration of the therapeutic agent in the system than can be provided by injection, oral formulation, or other typical delivery systems, thus minimizing possible toxic effects caused by the agent while maximizing therapeutic activity. Controlled delivery systems also advantageously decrease the frequency of injections required for a given therapeutic regimen and decrease the waste of expensive therapeutic agents by maintaining a steady-state concentrations of the agent within a desired narrow therapeutic window. The compositions are also useful in the treatment of isolated cells or tissues, where they may provide increased intracellular or intratissue delivery of a therapeutic agent or improved stability of the agent, for example over the time course of the treatment.

Porous silicon (pSi) refers to a nanostructured silicon-containing material that is typically formed by the etching of crystalline silicon wafers or other silicon-containing materials. See, e.g., Anglin et al. (2008) Adv. Drug Deliv. Rev. 60:1266, which is incorporated herein by reference in its entirety. As used herein, a silicon-containing material thus preferably includes elemental silicon (including crystalline and polycrystalline silicon), but may also include polysiloxanes, silanes, silicones, siloxanes, or combinations thereof. Porous silicon should be considered to encompass both the nanostructured material that results directly from the etching process, as well as any derivatives of that material, such as oxidized silicon or covalently-modified silicon, resulting from the further chemical modification of the etched porous silicon.

As just mentioned, porous silicon is typically prepared by either electrochemical or chemical stain etching of a silicon-containing material. Control of the etching process, for example, in the case of electrochemical etching, by controlling the current density, the type and concentration of dopant in the silicon wafer, the crystalline orientation of the wafer, and the electrolyte concentration, allows the size and morphology of the pores to be modulated as desired. Such modulation can result, for example, in microporous, mesoporous, or macroporous silicon.

Porous silicon was originally developed for use in optoelectronic devices after the discovery of its photoluminescent properties. Canham (1990) Appl. Phys. Lett. 57:1046. In recent years, however, pSi has gained interest as a carrier for the controlled release of drugs. Salonen et al. (2008) J. Pharm. Sci. 97:632; Chhablani et al. (2013) Invest. Ophthalmol. Vis. Sci. 54:1268; Kovalainen et al. (2012) Pharm. Res. 29:837. Conventional silicon-based compositions, such as, for example, mesoporous silica, are obtained by solution-phase reactions, for example by sol-gel or precipitation routes, with relatively little control over the microstructure of the resulting products. In contrast, significant control of the pSi microstructure is possible by tuning the electrochemical etching parameters used in its synthesis. Martinez et al. (2013) Biomaterials 34:8469; Hou et al. (2014) J. Control. Release 178:46. Porous silicon particles, including porous silicon particles oxidized in air at high temperatures, have been used for the delivery of therapeutic agents to the eye. See, for example, PCT International Publication No. WO 2006/050221 A2 and WO 2009/009563 A2, which is each incorporated herein by reference in its entirety for all purposes. Such particles were shown to deliver the agents over long periods of time with low toxicity upon intravitreal injection of the particles in rabbits.

Another useful feature of pSi is its readily modified surface chemistry. For example, methods such as thermal oxidation and thermal hydrosylilation may be used to optimize drug loading and release according to the properties of the drug payloads. Salonen et al. (2008) J. Pharm. Sci. 97:632; Anglin et al. (2008) Adv. Drug Deliv. Rev. 60:1266. It has been observed that certain chemistries can slow the degradation of the pSi matrix or enhance the release of poorly soluble active pharmaceutical ingredients (APIs). Salonen et al. (2005) J. Control. Release 108:362; Wang et al. (2010) Mol. Pharm. 7:227. Surface functionalization of pSi particles can be controlled in various ways, for example by differentially modifying inner pore walls and pore openings, as described in PCT International Publication No. WO 2014/130998 A1, which is incorporated by reference herein in its entirety.

Porous silicon is known to dissolve slowly in aqueous solutions at neutral pH, for example in normal body fluids, through a combination of oxidation of elemental Si and dissolution of the resulting silicic acids and ultimately orthosilicates. By controlling the rate and extent of this process, for example by modification of the surface of the pSi nanoparticles, the toxicity of the particles can be minimized significantly. For example, intravitreally injected pSi nanoparticles have been shown to be non-toxic and to reside safely in the rabbit vitreous for months before complete degradation and elimination from the eye. Cheng et al. (2008) Br. J. Ophthalmol. 92:705; Nieto et al. (2013) Exp. Eye Res. 116:161. See also U.S. Patent Publication No. 2010/0196435.

The particles and films of the instant disclosure, as described in more detail below, thus comprise a porous silicon core, which may also be referred to herein as a porous silicon “skeleton”. In some embodiments the porous silicon core comprises an etched crystalline silicon material, more specifically an electrochemically etched crystalline silicon material or a chemical stain etched crystalline silicon material. In embodiments, the porous silicon core comprises a microporous etched silicon material, for example a material comprising a plurality of pores with an average pore diameter of at most about 1 nm. In embodiments, the porous silicon core comprises a mesoporous etched silicon material, for example a material comprising a plurality of pores with an average pore diameter of from about 1 nm to about 50 nm. In embodiments, the porous silicon core comprises a macroporous etched silicon material, for example a material comprising a plurality of pores with an average pore diameter of from about 50 nm to about 1000 nm, or even larger.

In some embodiments, the porous silicon core of the instant particles and films has an open porosity from about 5% to about 95% based on the total volume of the material. In more specific embodiments, the porous silicon has an open porosity from about 20% to about 80%, or from about 40% to about 70% based on the total volume of the material. In some embodiments, the average pore diameter of the porous silicon of the instant compositions is from about 0.1 nm to about 1000 nm, from about 0.1 nm to about 1 nm, from about 0.1 nm to about 50 nm, from about 1 nm to about 50 nm, from about 1 nm to about 1000 nm, or from about 50 nm to about 1000 nm. In some embodiments, the average pore diameter is at least about 0.1 nm, at least about 0.5 nm, at least about 1 nm, at least about 50 nm, or even larger. In some embodiments, the average pore diameter is at most about 1000 nm, at most about 100 nm, at most about 50 nm, at most about 1 nm, or even smaller.

The porous silicon core of the instant compositions can be in the form of a film or a particle. In particular, the thickness of the instant particles and films preferably ranges from about 5 nm to about 1000 microns, from about 10 nm to about 100 microns, or from about 100 nm to about 30 microns. Accordingly, the particles and films can have a thickness of at least about 5 nm, at least about 10 nm, at least about 100 nm, or even thicker. Likewise, the particles and films can have a thickness of at most about 1 mm, at most about 100 microns, at most about 30 microns, or even thinner. In embodiments where the porous silicon core is in the form of a particle, the average diameter of the porous silicon core preferably ranges from about 1 nm to about 1 cm, from about 3 nm to about 1000 microns, from about 10 nm to about 300 microns, from about 10 nm to about 100 microns, or from about 1 micron to about 50 microns. In some embodiments, the average particle diameter is at least about 1 nm, at least about 3 nm, at least about 10 nm, at least about 100 nm, at least about 1 micron, or even larger. In some embodiments, the average particle diameter is at most about 1 cm, at most about 1000 microns, at most about 300 microns, at most about 100 microns, at most about 50 microns, or even smaller.

In certain embodiments, the porous silicon core of the instant compositions is at least partially oxidized. Oxidation of elemental silicon to silicon dioxide in the porous silicon compositions of the instant disclosure may increase the stability of the compositions, decrease the toxicity of the compositions, and/or provide improved dissolution properties. Exemplary methods for oxidizing the porous silicon of the instant compositions is provided in detail below in the methods of preparation. The oxidized porous silicon materials of any of those methods, whether completely oxidized or partially oxidized, find utility in the instant compositions. In some cases, it may be desirable to oxidize the porous silicon core by substituting nitrate, nitrite, gluconate, or other suitable anions for the chloride of the metal salts used in the methods of preparation described below. Metal nitrates or metal nitrites can oxidize porous silicon more quickly than metal chlorides due to the oxidizing nature of the nitrate and nitrite ions. Fry et al. (2014) Chem. Mater. 26:2758.

It should be understood that the term “porous silicon oxide” refers to a substance containing silicon and oxygen of general stoichiometric formula SiOₓ, where x can be as small as 0.01 and as large as 2, and that “porous silicon” refers to a substance that is composed of elemental silicon (either in its crystalline or amorphous state), with a surface containing hydrogen, oxygen, or carbon-containing species. Furthermore, the terms “porous silicon” or “porous silicon oxide” refer to materials that containing micropores, mesopores, or macropores, or combinations of any two or all three pore types. It should also be understood that the surface of the porous materials, including the surface of the inner pore walls, may contain hydrogen, oxygen, or carbon-containing species.

Exemplary compositions comprising porous silicon and methods of preparing those compositions are described in detail in, e.g., U.S. Patent Publication Nos. 2005/0042764; 2005/0009374; 2007/0148695; 2007/0051815; 2009/0208556; and 2010/0196435; each of which is hereby incorporated by reference herein in its entirety.

In some embodiments, the porous silicon core of the instant disclosure has been covalently modified. In specific embodiments, the covalent modification is on the surface of the porous silicon core. Examples of porous silicon that has been modified by surface modification, such as alkylation and in particular thermal hydrosilylation, are described in Cheng et al. (2008) Br. J. Ophthalmol. 92:705 and PCT International Publication No. WO2014/130998 A1. Such materials were found to display good biocompatability when used as a delivery system for therapeutic agents.

As mentioned above, porous silicon is known to dissolve slowly in aqueous solutions at neutral pH. The mechanism of degradation of porous silicon involves oxidation of the silicon skeleton to form silicon oxide (eq. 1), and dissolution of the resulting oxide phase to water-soluble ortho-silicic acid (Si(OH)₄) or its congeners (eq. 2). See Sailor, Porous silicon in practice: preparation, characterization and applications. (John Wiley & Sons, 2012).

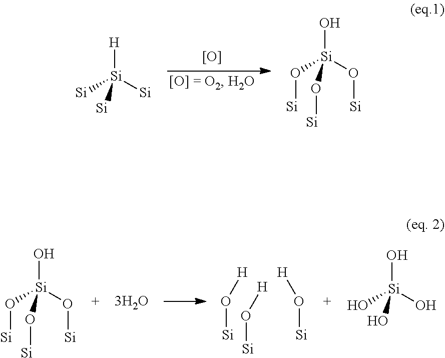

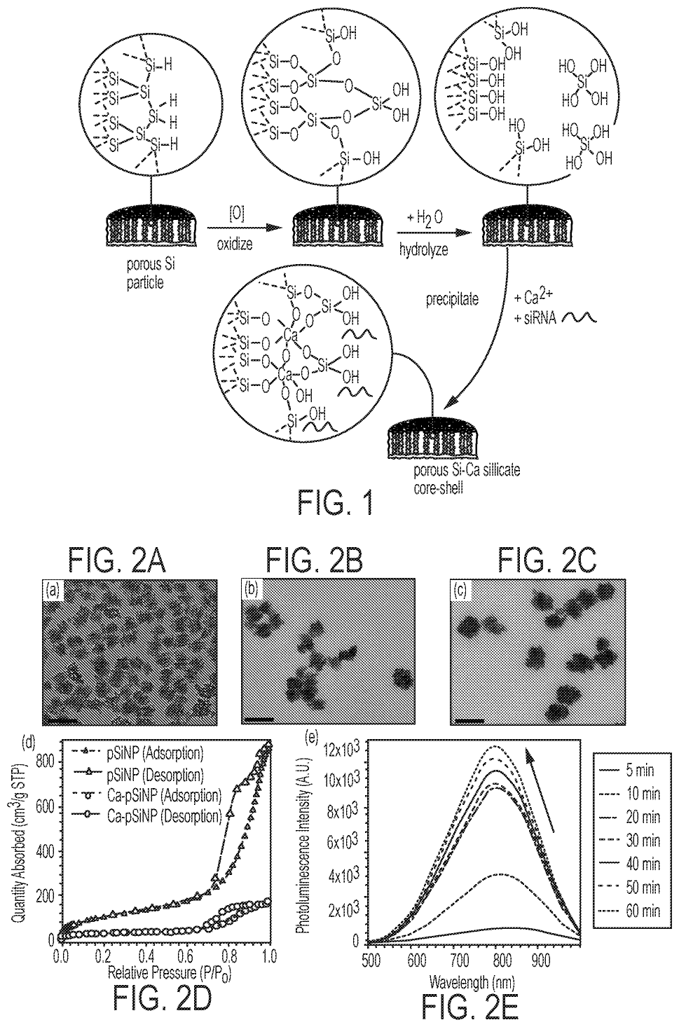

It has been advantageously discovered that reaction of the silicic acid produced by dissolution of porous silicon or porous silicon oxide with a high concentration of a metal salt results in the formation of an insoluble metal salt that includes the anions orthosilicate (SiO₄⁴⁻), metasilicate (SiO₃²⁻), or their congeners, referred to herein as “silicate”. Without intending to be bound by theory, the insoluble silicate salt is thought to act as a protective shell that impedes further dissolution of the porous silicon or porous silicon oxide skeleton. Furthermore, the formation of the insoluble salt serves to block the pore openings of the material, such that a substance previously loaded in the pores can become trapped. See FIG. 1 for an illustration with an siRNA therapeutic agent. Although it has been demonstrated previously that exposing porous silicon to solutions containing relatively low concentrations of aqueous calcium and phosphate leads to the formation of a surface layer of hydroxyapatite (Li et al. (1998) J. Am. Chem. Soc. 120:11706), the formation of an insoluble silicate in the presence of high concentrations of metal salts has not been demonstrated nor has the surprising efficacy of these reactions in trapping substantial quantities of payload molecules. While it is known from the chemistry of cement that calcium oxide reacts with silica to form calcium silicate (Minet et al. (2006) J. Mater. Chem. 16:1379), and that mixing homogeneous precursors, such as an aqueous silicate solution and an calcium ion solution, can generate precipitates and nanoparticles (Wu et al. (2012) J. Mater. Chem. 22:16801; Wu et al. (2010) Adv. Mater. 22:749; Li et al. (2007) J. Biomed. Mater. Res. B. 83B:431; Saravanapavan et al. (2003) J. Noncrystalline Solids 318:1; Kokubo et al. (2003) Biomaterials 24:2161; and Salinas et al. (2001) J. Sol-Gel Sci. Techn. 21:13), it has not been previously demonstrated that aqueous metal salt solutions reacting with nanostructured porous silicon can yield a core/shell nanostructure. In addition, the core/shell structures of the present compositions display unique properties that differ from those of materials prepared by the above homogeneous routes, and the preparative methods described herein advantageously allow the loading and subsequent slow release of therapeutic agents. Furthermore, the ability of core-shell structures to enhance the intensity and persistence of photoluminescence from the luminescent silicon domains in porous silicon has been demonstrated (Joo et al. (2014) Adv. Funct. Mater. 24:5688), and it is demonstrated herein that these new shells provide similar improvement to the intrinsic photoluminescence properties of porous silicon.

The porous silicon particles and films of the instant disclosure thus preferably comprise a layer on the surface of the porous silicon core that comprises a metal silicate. As described above, this layer may also be referred to in some instances as a “shell”. In some embodiments, the metal silicate is a divalent, trivalent, or tetravalent metal silicate. More specifically, the metal silicate is a divalent silicate. For example, the divalent metal silicate can be a calcium silicate, a magnesium silicate, a manganese silicate, a copper silicate, a zinc silicate, a nickel silicate, a platinum silicate, or a barium silicate. In specific embodiments, the divalent metal silicate is a calcium silicate or a magnesium silicate. Even more specifically, the divalent metal silicate is a calcium silicate. In other specific embodiments, the metal silicate is a trivalent or tetravalent metal silicate. Exemplary trivalent or tetravalent metal silicates having utility in the porous silicon particles and films of the instant disclosure include zirconium silicates, titanium silicates, and bismuth silicates. In some embodiments, the layer on the surface of the porous silicon core comprises a combination of metal silicates, including any of the above-listed exemplary metal silicates in any combination.

While porous silicon or porous silicon oxide nanostructures are readily configured to accept a therapeutic agent, a diagnostic agent, or another beneficial substance (also referred to as a “payload”) (Salonen et al. (2008) J. Pharm. Sci. 97:632; Anglin et al. (2008) Adv. Drug Deliv. Rev. 60:1266), the premature release of these payloads, either prior to or post-administration, can be undesirable for the intended purpose. Additionally, the degradation of porous silicon or porous silicon oxide in aqueous conditions can pose significant problems in applications involving sustained drug delivery (Salonen et al. (2008) J. Pharm. Sci. 97:632; Anglin et al. (2008) Adv. Drug Deliv. Rev. 60:1266), in vivo or in vitro imaging (Joo et al. (2014) Adv. Funct. Mater. 24:5688; Gu et al. (2013) Nat. Commun. 4:2326; Park et al. (2009) Nat. Mater. 8:331), and biosensors (Jane et al. (2009) Trends Biotechnol. 27:230), which has led to the synthesis of various “core-shell” types of structures, where an inner core of silicon or silicon oxide (in the porous skeleton) is surrounded with a shell of more stable silicon oxide (Joo et al. (2014) Adv. Funct. Mater. 24:5688), titanium oxide (Betty et al. (2011) Prog. Photovoltaics 19:266; Li et al. (2014) Biosens. Bioelectron. 55:372), carbon (Tsang et al. (2012) ACS Nano 6:10546), or other kinetically stable substance (Buriak (2002) Chem. Rev. 102:1271). Under the appropriate conditions, formation of the shell can trap a substance previously loaded in the pores, providing a slow releasing formulation (Fry et al. (2014) Chem. Mater. 26:2758). Fusogenic liposome-coated porous silicon nanoparticles comprising a core-shell structure with cargo molecules physically trapped within the porous silicon-containing core material are described in U.S. Provisional Application No. 62/190,705, filed on Jul. 9, 2015, and PCT International Publication No. WO 2017/008059 A1, both of which are incorporated herein by reference in their entireties.

The presence of metal ions, such as calcium ions, in the instant compositions, can additionally be beneficial to tissues, as the ions can sequester residual fluoride ions that may be present in the formulations. The porous silicon and porous silicon oxide materials used in the subject compositions are typically prepared by an electrochemical etch in fluoride-containing electrolytes, and this process can leave trace amounts of fluoride in the porous matrix (Koynov et al. (2011) Adv. Eng. Mater. 13:B225). Fluoride can be highly toxic to tissues (particularly to sensitive tissues, such as the eye). Because of the very low solubility product of calcium fluoride and other metal fluorides in aqueous solution, however, the use of high concentrations of metal ions in the core-shell synthesis may serve an added benefit of reacting with residual fluoride in the formulation, thus neutralizing the fluoride and its deleterious in vivo effects.

In some embodiments, the layer on the surface of the porous silicon core has a thickness of between 1 and 90 percent, between 5 and 60 percent, or between 10 and 40 percent of the average diameter or thickness of the core.

In preferred embodiments, the metal silicate of the layer on the surface of the porous silicon core is chemically linked to the porous silicon core.

The compositions of the instant disclosure additionally comprise a therapeutic agent, preferably contained within the etched pores of the porous silicon particles or films. It should be understood that the term therapeutic agent should be construed broadly to encompass any agent capable of having a therapeutic effect on a subject, tissue, or cell in need of treatment. Therapeutic agents include biological polymers, such as nucleic acids, carbohydrates, and proteins, as well as lipids and any other naturally-occurring molecules, including primary and secondary metabolites. Therapeutic agents may also include any derivatives or otherwise modified versions of the above molecules that provide a therapeutic activity. Indeed, the therapeutic agent may have a structure that is partially or entirely non-natural. The therapeutic agent may be purified from natural sources, may be prepared using semi-synthetic methods, or may be prepared entirely by synthetic approaches. The therapeutic agent may be provided as a pharmaceutically acceptable salt form and may be formulated together with a pharmaceutically acceptable excipient or other agent having non-therapeutic effects. In some situations it may be advantageous to combine more than one therapeutic agent in a single composition of the disclosure or even within a single porous silicon particle or film.

The therapeutic agents usefully included in the instant compositions include, without limitation, ACE inhibitors, actin inhibitors, analgesics, anesthetics, anti-hypertensives, anti polymerases, antisecretory agents, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasitics, anti-Parkinson agents, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, hormones, hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, opioids, prostaglandins, remodeling inhibitors, statins, steroids, thrombolytic agents, tranquilizers, vasodilators, and/or vasospasm inhibitors.

In some embodiments, the therapeutic agent is a nucleic acid or a nucleic acid analogue, for example but not limited to a deoxyribonucleic acid (DNA) or a ribonucleic acid (RNA), for example a small interfering RNA (siRNA), a messenger RNA (mRNA), a transfer RNA (tRNA), a microRNA (miRNA), a small temporal RNA (stRNA), a small hairpin RNA (shRNA), a modified mRNA (mmRNA), or analogues or combinations thereof. In some embodiments, the therapeutic agent is a nucleic acid analogue, for example but not limited to antisense nucleic acids, oligonucleic acids, or oligonucleotides, peptide nucleic acid (PNA), pseudo-complementary PNA (pcPNA), locked nucleic acid (LNA), or derivatives or analogues thereof. In preferred embodiments, the therapeutic agent is an siRNA.

It would be understood by those of ordinary skill in the art that there may be advantages to the delivery of negatively-charged therapeutic agents, such as nucleic acid agents, in the instant compositions, in view of the formulation of these agents with a metal silicate layer on the surface of the instant pSi nanoparticles or nanofilms. Without intending to be bound by theory, the metal component of the composition may neutralize the anionic charge of the nucleic acid therapeutic agent component, thus improving the loading capacity of the instant materials.

In some embodiments, the therapeutic agent is a protein or peptide, for example an antibody or protein biologic, a peptidomimetic, an aptamer, or a variant thereof.

In some embodiments, the therapeutic agent is an antibiotic, such as, for example, a lipopeptide (e.g., daptomycin), a glycylcycline (e.g., tigecycline), an oxazolidinone (e.g., linezolid), a lipiarmycin (e.g., fidaxomicin), a penicillin, a cephalosporin, a polymyxin, a rifamycin, a quinolone, a sulfonamide, a macrolide, a lincosamide, a tetracycline, a glycopeptide (e.g., vancomycin), and the like.

In some embodiments, the therapeutic agent is a small-molecule hydrophobic therapeutic agent. Many therapeutic agents, in particular, hydrophobic therapeutic agents, are more efficiently delivered to biological systems in their non-crystalline forms, i.e., as amorphous forms. Indeed, the formulation of hydrophobic therapeutic agents into amorphous forms is considered a promising strategy for increasing dissolution properties and thus increasing bioavailability. Due to the high internal energy of the amorphous form of an active agent, however, pure amorphous agents often recrystallize rapidly to their lower energy, crystalline state, which typically has low solubility. It is therefore desirable to formulate hydrophobic therapeutic agents such that the amorphous state is stabilized.

Without intending to be bound by theory, it is believed that the pore surfaces of the instant porous silicon particles and films, in particular porous silicon materials where the pore surface has been modified, may stabilize the amorphous form of a therapeutic agent by strong molecular interactions between the agent and the pore surface. These interactions may prevent the drug from recrystallization and thus may ensure efficient release and increased bioavailability of the agent.

Examples of small-molecule therapeutic agents usefully incorporated into the instant particles and films therefore include hydrophobic therapeutic agents. In specific embodiments, the hydrophobic agent is rapamycin, paclitaxel, daunorubicin, doxorubicin, or an analogue of any of these agents. In preferred embodiments, the agent is rapamycin (also known as sirolimus) or a rapamycin analogue. Non-limiting examples of rapamycin analogues include, for example, everolimus, zotarolimus, biolimus A9, temsirolimus, myolimus, novolimus, tacrolimus, or pimecrolimus.

Covalently modified versions of rapamycin may be usefully included in the instant compositions without limitation. For example, U.S. Pat. Nos. 4,316,885 and 5,118,678 report carbamates of rapamycin. U.S. Pat. No. 4,650,803 reports water-soluble prodrugs of rapamycin. U.S. Pat. No. 5,100,883 reports fluorinated esters of rapamycin. U.S. Pat. No. 5,118,677 reports amide esters of rapamycin. U.S. Pat. No. 5,130,307 reports aminoesters of rapamycin. U.S. Pat. No. 5,346,893 reports sulfonates and sulfamates of rapamycin. U.S. Pat. No. 5,194,447 reports sulfonylcarbamates of rapamycin. U.S. Pat. No. 5,446,048 reports rapamycin oximes. U.S. Pat. No. 6,680,330 reports rapamycin dialdehydes. U.S. Pat. No. 6,677,357 reports rapamycin 29-enols. U.S. Pat. No. 6,440,990 reports O-alkylated rapamycin derivatives. U.S. Pat. No. 5,955,457 reports water soluble rapamycin esters. U.S. Pat. No. 5,922,730 reports alkylated rapamycin derivatives. U.S. Pat. No. 5,637,590 reports rapamycin amidino carbamates. U.S. Pat. No. 5,504,091 reports biotin esters of rapamycin. U.S. Pat. No. 5,567,709 reports carbamates of rapamycin. U.S. Pat. No. 5,362,718 reports rapamycin hydroxyesters. These rapamycin derivatives, and others, may be included in the instant compositions.

The amount of a therapeutic agent incorporated into the compositions of the instant disclosure will depend upon the desired release profile, the concentration of the therapeutic agent required for a biological effect, and the length of time that the therapeutic agent has to be released for treatment. There is no upper limit on the amount of therapeutic agent incorporated into the instant compositions except for that of an acceptable solution or dispersion viscosity for injection through a syringe needle or other appropriate delivery device. The lower limit of therapeutic agent incorporated into the instant compositions is dependent upon the activity of the therapeutic agent and the length of time needed for treatment. Specifically, in one embodiment of the present invention, the composition is formulated to provide a one month release of therapeutic agent. In such an embodiment, the therapeutic agent is preferably present in about 0.1 wt. % to about 50 wt. %, preferably about 2 wt. % to about 25 wt. % of the composition. Alternatively, in another embodiment of the present disclosure, the composition is formulated to provide a three month delivery of therapeutic agent. In such an embodiment, the therapeutic agent is preferably present in about 0.1 wt. % to about 50 wt. %, preferably about 2 wt. % to about 25 wt. % of the composition. Alternatively, in another embodiment of the present disclosure, the composition is formulated to provide a six month delivery of therapeutic agent. In such an embodiment, the therapeutic agent is preferably present in about 0.1 wt. % to about 50 wt. %, preferably about 2 wt. % to about 25 wt. % of the composition. The composition releases the therapeutic agent contained therein at a controlled rate until the composition is completely dissolved.

In some embodiments, the therapeutic agent is not covalently associated with the particle or film comprising a porous silicon core. In some embodiments, the therapeutic agent is contained within the pores of the porous silicon core.

In certain embodiments, the compositions for delivering a therapeutic agent of the instant disclosure further comprise a targeting agent and/or a cell-penetrating agent. In these embodiments, the particles of the instant compositions are preferably sized to transport the therapeutic agent from the site of administration to the site of desired therapeutic effect.

Targeting agents suitable for use in the instant disclosure thus include agents that can target the particles of the instant compositions to a specific tissue within a treated subject. In particular, the targeting agent may, for example, comprise a peptide or other moiety that binds to a cell surface component, such as a receptor or other surface protein or lipid found on the cell to be targeted. Examples of suitable targeting agents are short peptides, protein fragments, and complete proteins. Ideally, the targeting agent should not interfere with uptake of the particle by the targeted cell. Targeting agents may comprise in some embodiments no more than 100 amino acids, for example no more than 50 amino acids, no more than 30 amino acids, or even no more than 10, 5, or 3 amino acids.

Targeting agents can be selected to target the particles to a particular cell or tissue type, for example particles can be targeted to muscle, brain, liver, pancreas, or lung tissue, or to macrophages or monocytes. Alternatively, a targeting agent can be selected to target the particles to specific cells within a diseased tissue, such as, for example, tumor cells, diseased coronary artery cells, brain cells affected by Alzheimer's disease, bacterial cells, or virus particles. In preferred embodiments of the instant disclosure, the targeting agent is selective for neuronal tissue, such as, for example, for brain tissue.

Specific examples of targeting agents include a muscle-specific peptide, discovered by phage display to target skeletal muscle (Flint et al. (2005) Laryngoscope 115:1930), a 29-residue fragment of rabies virus glycoprotein that binds to the acetylcholine receptor (Lentz (1990) J. Mol. Recognit. 3:82), a fragment of neural growth factor that targets its receptor to target neurons, and a secretin peptide that binds to the secretin receptor and that can be used to target biliary and pancreatic epithelia, for example in cystic fibrosis (Zeng et al. (2004) J. Gene Med. 6:1247 and McKay et al. (2002) Mol. Ther. 5:447). As an alternative, immunoglobulins and their variants, including scFv antibody fragments, can also be used as targeting agents to bind to specific antigens, such as VEGFR or other surface proteins, on the surface of targeted cells or tissues. As yet another alternative, receptor ligands can used as targeting agents to target the particles to the surface of cells or tissues expressing the targeted receptor. In specific embodiments, the targeting agent of the instant compositions is a neuronal targeting agent, such as, for example, a peptide sequence from the rabies virus glycoprotein (RVG).

Cell-penetrating agents of the instant disclosure are also known as internalization agents or cell membrane transduction agents. In specific embodiments, the cell-penetrating agents are cell-penetrating peptides or proteins. These agents include the well-known class of relatively short (e.g., 5-30 residue, 7-20 reside, or even 9-15 residue) peptides that allow certain cellular or viral proteins to traverse membranes, but other classes are known. See, e.g., Milletti (2012) Drug Discov. Today 17: 850. Exemplary peptides in the original class of cell-penetrating peptides typically have a cationic charge due to the presence of relatively high levels of arginine and/or lysine residues which are believed to facilitate the passage of the peptides across cellular membranes. In some cases, the peptides have 5, 6, 7, 8, or even more arginine and/or lysine residues. Exemplary cell-penetrating peptides include penetratin or antennapedia PTD and variants, TAT, SynB1, SynB3, PTD-4, PTD-5, FHB Coat-(35-49), BMV Gag-(7-25), HTLV-II Rex-(4-16), D-Tat, R9-Tat, Transportan, MAP, SBP, FBP, MPG and variants, Pep-1, Pep-2, and various periodic sequences, including polyarginines, polylysines, and their variants. See http://crdd.osdd.net/raghava/cppsite/index.html and http://cell-penetrating-peptides.org for additional examples of cell-penetrating peptides useful in the instant compositions.

Several proteins, lectins, and other large molecules, for example plant and bacterial protein toxins such as ricin, abrin, modeccin, diphtheria toxin, cholera toxin, anthrax toxin, heat labile toxins, Pseudomonas aeruginosa exotoxin A (ETA), or fragments thereof, also display cell-penetrating properties and can be considered cell-penetrating agents for purposes of this disclosure. Other exemplary cell-penetrating agents are described in Temsamani et al. (2004) Drug Discov. Today 9:1012; De Coupade et al. (2005) Biochem J. 390:407; Säälik et al. (2004) Bioconjug. Chem. 15:1246; Zhao et al. (2004) Med. Res. Rev. 24:1; and Deshayes et al. (2005) Cell. Mol. Life Sci. 62:1839; all of which are incorporated by reference herein in their entireties.

In some embodiments, the cell-penetrating agent, for example a cell-penetrating peptide, can be derivatized, for example by acetylation, phosphorylation, lipidation, pegylation, and/or glycosylation, to improve the binding affinity of the agent, to improve the ability of the agent to be transported across a cell membrane, or to improve stability. In specific embodiments, the cell-penetrating agent is lipidated, for example by myristoylation, palmitoylation or attachment of other fatty acids preferably with a chain length of 10-20 carbons, such as lauric acid and stearic acid, as well as by geranylation, geranylgeranylation, and other types of isoprenylation. In more specific embodiments, the cell-penetrating agent is myristoylated.

In specific embodiments, the cell-penetrating agent is Transportan, and more specifically is a lipidated Transportan. In even more specific embodiments, the cell-penetrating agent is myristoylated Transportan.

In some embodiments, the compositions of the disclosure comprise both a targeting agent and a cell-penetrating agent, whereas in other embodiments comprise either a targeting agent or a cell-penetrating agent. For example, when the compositions are used for the treatment of an animal subject, for example a human subject, and in particular when the treatment is a systemic treatment, it may be advantageous for the compositions to include both a targeting agent and a cell-penetrating agent. When the administration is directly to a particular tissue of an animal subject, it may not be necessary for the compositions to include a targeting agent. When the compositions are used for other purposes, for example when the compositions are directed to extracellular targets, it may not be necessary for the compositions to include a cell-penetrating agent. In some cases, for example where the compositions are administered directly to isolated cells or tissues, the compositions may include neither a targeting agent nor a cell-penetrating agent, as would be understood by those of ordinary skill in the art.

Exemplary compositions comprising porous silicon particles according to the instant disclosure are described in Kang et al. (2016) Adv. Mater. 28:7962, which is incorporated by reference herein in its entirety.

### Methods of Preparing Porous Silicon Particles Comprising a Metal Silicate Layer

In another aspect, the instant disclosure provides methods of preparing the above-described porous silicon particles and films. In particular, the disclosure provides methods of loading and protecting one or more therapeutic agents in the pores and/or surface layers of such materials. In some embodiments, the methods comprise the steps of providing a porous silicon precursor particle or film, and treating the porous silicon precursor particle or film with an aqueous solution comprising the therapeutic agent and a metal salt. In preferred embodiments, the methods are applied to the treatment of a porous silicon precursor particle.

The term “precursor particle” or “precursor film” is used herein simply to distinguish the particles and films used in the methods of preparation from the products of those methods.

In specific embodiments, the porous silicon precursor materials used in the instant methods of preparation have the chemical and structural features of the particles and films described above. For example, the porous silicon precursor material may have a thickness ranging from about 5 nm to about 1000 microns, from about 10 nm to about 100 microns, or from about 100 nm to about 30 microns. In embodiments where the porous silicon precursor material is in the form of a particle, the particle may have a average size ranging from about 1 nm to about 1 cm, from about 3 nm to about 1000 microns, from about 10 nm to about 300 microns, from about 10 nm to about 100 microns, or from about 1 micron to about 50 microns.

The instant porous silicon compositions may be prepared from porous silicon precursor films and precursor particles by known methods. See generally, for example, Sailor, Porous silicon in practice: preparation, characterization and applications (John Wiley & Sons, 2012) and Qin et al. (2014) Part. Part. Syst. Char. 31:252. Specifically, a silicon wafer may be electrochemically etched with, for example, a 3:1 48%-HF:EtOH solution, with proper current density to obtain determined particle size, porosity, and pore size. The layer of etched porous silicon may be removed from the wafer, for example by applying a low current density pulse in dilute aqueous HF. For the preparation of pSi nanoparticles (pSiNPs), perforations along the etched planes may be introduced by short periodic pulses of high current (e.g., 370 mA/cm2, 0.4 sec) during a long low-current etch (e.g., 40 mA/cm2, 1.8 sec), thus generating layers of alternating high and low porosity (Qin et al. (2014) Part. Part. Syst. Char. 31:252). The layer of porous silicon may be removed from the wafer to form films, and the freestanding films may be fractured for example by overnight ultrasonication, to generate monodispersed pSi nanoparticles. For the preparation of pSi microparticles (pSiMPs), an etching current ranging from, for example, 20 to 100 mA/cm² may be applied at a period of, for example, 4 sec and 2.7 sec per cycle to form a composite sinusoidal structure with stop bands at approximately 450 and 560 nm. The freestanding films may be fractured by ultrasonication for 5-7 min to generate pSi microparticles of the desired size (e.g., 20×60×60 μm).

It will be apparent to those skilled in the art that other current-time waveforms may be used to generate electrochemically etched porous silicon material. For example, a single constant current for a predetermined time period or a sinusoidal current-time waveform may be used in such approaches. Alternatively, a chemical stain etch may be substituted for the electrochemical etch described above to produce the porous silicon core. See Sailor, Porous silicon in practice: preparation, characterization and applications (John Wiley & Sons, 2012). In yet another alternative, the porous silicon core may be produced by the chemical reduction of a nanostructured silicon oxide. See Batchelor et al. (2012) Silicon 4:259. Stain etching typically uses silicon powder instead of silicon wafers as the silicon precursor and a chemical oxidant instead of an electrical power supply to drive the electrochemical reaction.

In some embodiments, the porous silicon precursor material may be oxidized or partially oxidized. In specific embodiments, the porous silicon precursor material may be thermally oxidized, for example at a temperature of at least 150° C., at least 200° C., at least 300° C., at least 400° C., at least 500° C., at least 600° C., at least 700° C., at least 800° C., or even higher. In some embodiments, the porous silicon precursor material may be oxidized at a temperature of from about 300° C. to about 1000° C., at a temperature of from about 400° C. to about 800° C., or at a temperature of from about 500° C. to about 700° C. In preferred embodiments, the thermal oxidation is performed in air.

In certain embodiments, the porous silicon precursor material of the instant methods may be oxidized in solution, for example by suspending the porous silicon material in a solution comprising an oxidizing agent. For example, the solution used to oxidize the porous silicon material may comprise water, borate, tris(hydroxymethyl)aminomethane, dimethyl sulfoxide, nitrate salts, or any other suitable oxidizing agent or combination of agents.

As previously described, the solution used to prepare the instant compositions typically comprises a metal salt. In specific embodiments, the solution comprises a concentration of metal salt of at least 0.1 molar, 0.3 molar, 0.5 molar, 1 molar, 2 molar, 3 molar, or even higher. In some specific embodiments, the metal salt is a divalent, trivalent, or tetravalent metal salt. More specifically, the metal salt is a divalent metal salt. For example, the divalent metal salt can be a calcium salt, a magnesium salt, a manganese salt, a copper salt, a zinc salt, a nickel salt, a platinum salt, or a barium salt. In specific embodiments, the divalent metal salt is a calcium salt or a magnesium salt. Even more specifically, the divalent metal salt is a calcium salt. In other specific embodiments, the metal salt is a trivalent or tetravalent metal salt. Exemplary trivalent or tetravalent metal salts having utility in the preparative methods of the instant disclosure include zirconium salts, titanium salts, and bismuth salts. In some embodiments, the methods of preparation utilize a combination of metal salts, including any of the above-listed exemplary metal salts in any combination. In some embodiments, the step of treating the porous silicon precursor particle or film with an aqueous solution comprising the therapeutic agent and the metal salt is performed in a single step.

In some embodiments, the therapeutic agent used in treating the porous silicon particle or film is any of the therapeutic agents described in detail above. For example, the agent may be a small-molecule drug, a vitamin, an imaging agent, a protein, a peptide, a nucleic acid, an oligonucleotide, an aptamer, or a mixture thereof. More specifically, the therapeutic agent may be an oligonucleotide, such as a DNA, an RNA, an siRNA, or a micro-RNA. In embodiments where the therapeutic agent is an oligonucleotide, the therapeutic agent may preferably be a ribonucleotide or even an siRNA.

The methods of preparation may additionally comprise the step of coupling the porous silicon precursor particle or film with a targeting agent. More specifically, the targeting agent may be a neuronal targeting agent or any of the specific targeting agents described above. The methods of preparation may alternatively or additionally comprise the step of coupling the porous silicon precursor particle with a cell-penetrating agent, more specifically a lipidated peptide, or any of the specific cell-penetrating agents described above. In specific embodiments, the methods of preparation may additionally comprise the step of coupling the porous silicon precursor particle with a targeting agent and a cell-penetrating agent. Exemplary targeting agents and cell-penetrating agents are described in detail above.

### Pharmaceutical Compositions

In another aspect, the instant disclosure provides pharmaceutical compositions comprising a particle- or film-comprising composition of the disclosure and a pharmaceutically acceptable carrier. The pharmaceutical composition may be in dosage unit form such as tablet, capsule, sprinkle capsule, granule, powder, syrup, suppository, injection, or the like. The composition may also be present in a transdermal delivery system, e.g., a skin patch.

The phrase “pharmaceutically acceptable” is employed herein to refer to those compounds, materials, compositions, and/or dosage forms that are, within the scope of sound medical judgment, suitable for use in contact with the tissues of animal subjects, including human subjects, without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material, involved in carrying or transporting the subject particle-comprising compositions from one organ, or portion of the body, to another organ, or portion of the body. Each carrier must be “acceptable” in the sense of being compatible with the other ingredients of the formulation and not injurious to the patient, as is understood by those of ordinary skill in the art. Some examples of materials that can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. See Remington: The Science and Practice of Pharmacy, 20th ed. (Alfonso R. Gennaro ed.), 2000. When the therapeutic agent of the instant compositions is a nucleic acid, in particular a ribonucleic acid, the pharmaceutically acceptable carrier should preferably be substantially free of nucleases, such as, for example, ribonucleases.

A pharmaceutical composition containing a particle-comprising composition of the instant disclosure may be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally, or vaginally (for example, as a pessary, cream, or foam); parenterally (including intramuscularly, intravenously, subcutaneously, or intrathecally as, for example, a sterile solution or suspension); nasally; intraperitoneally; subcutaneously; transdermally (for example as a patch applied to the skin); or topically (for example, as a cream, ointment or spray applied to the skin). The composition may also be formulated for inhalation. In certain embodiments, a particle-comprising composition of the instant disclosure may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

### Methods of Treatment

The instant compositions find particular utility in methods where the delivery of a therapeutic agent is usefully provided in a controlled manner. For example, and as described above for pharmaceutical compositions, the methods may be of use in the delivery of a therapeutic agent orally, sublingually, anally, rectally, vaginally, parenterally, nasally, intraperitoneally, subcutaneously, transdermally, topically, by inhalation, or by any other suitable mode of administration, as would be understood by those of ordinary skill in the art. In preferred embodiments, the methods of treatment target therapeutic agents to neuronal tissue, in particular to the brain.

As described above, the compositions of the instant disclosure may be luminescent, and this property may facilitate the monitoring of subjects that are administered these compositions. Accordingly, in some embodiments, the methods of treatment further comprise the step of monitoring the subject or tissues isolated from the subject. In view of the photoluminescent properties of some of the compositions of the instant disclosure, in specific embodiments, the monitoring step is an optical monitoring step.

It will be readily apparent to one of ordinary skill in the relevant arts that other suitable modifications and adaptations to the compositions, methods, and applications described herein may be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

Self-Sealing Porous Silicon-Calcium Silicate Core-Shell Nanoparticles for Targeted siRNA Delivery to the Injured Brain

A single-step procedure to simultaneously load and protect high concentrations of siRNA in porous silicon nanoparticles (pSiNPs) is presented. Treatment of pSiNPs with an aqueous solution containing siRNA and calcium chloride generates core-shell nanostructures consisting of an siRNA-loaded pSiNP core infiltrated with an surface layer of calcium silicate (Ca-pSiNPs). The source of silicate in the shell derives from local dissolution of the pSi matrix. Without intending to be bound by theory, it is understood that in solutions containing high concentrations of calcium (II) ion, Ca₂SiO₄ formation occurs primarily at the nanoparticle surface and is self-limiting. Accordingly, it is understood that the insoluble calcium silicate shell slows the degradation of the pSiNP core and prolongs delivery of the siRNA payload, resulting in more effective gene knockdown in vitro and in vivo. Formation of the calcium silicate shell results in an increase in the external quantum yield of photoluminescence from the porous silicon core from 0.1 to 21%, presumably due to the electronically passivating nature of the silicate shell. Attachment of two functional peptides that incorporate a sequence derived from the rabies virus glycoprotein (RVG) as a neuronal targeting peptide and myristoylated Transportan (mTP) as a cell penetrating moiety to the Ca-pSiNPs yields a construct that shows improved gene silencing in vitro and improved delivery in vivo.

A significant limitation in efficacy of small molecule, protein, and nucleic acid-based therapeutics is bioavailability. Molecules with low solubility may not enter the blood stream or other bodily fluids at therapeutically effective concentrations (Muller et al. (2001) Adv. Drug Deliver. Rev. 47:3; Kataoka et al. (2012) Pharm. Res.-Dordr. 29:1485; Kipp (2004) Int. J. Pharm. 284:109), and more soluble therapeutics may undergo rapid clearance from the circulatory system by various biological processes before reaching the intended tissues (Chonn et al. (1992) J. Biol. Chem. 267:18759; Pirollo et al. (2008) Trends Biotechnol. 26:552; Gabizon et al. (1988) P. Natl Acad. Sci. USA 85:6949). Loading of therapeutics into porous or hollow nanostructures has emerged as a means to control the concentration-time relationship of drug delivery and thus to improve therapeutic efficacy. Lou et al. (2008) Adv. Mater. 20:3987; Anglin et al. (2008) Adv. Drug Deliver. Rev. 60:1266. Much work in nanostructured carriers for drugs has been based on “soft” particles such as liposomes and polymer conjugates (Gu et al. (2011) Chem. Soc. Rev. 40:3638; Nishiyama et al. (2006) Pharmacol. Therapeut. 112:630), or more rigid porous inorganic materials such as mesoporous silicon or silicon oxide (Park et al. (2009) Nat. Mater. 8:331; Wu et al. (2008) ACS Nano 2:2401; Godin et al. (2010) J. Biomed. Mater. Res. A 94a:1236). Mesoporous silicon and silicon oxide are inorganic and biodegradable materials that have been well studied for drug delivery applications. Anglin et al. (2008) Adv. Drug Deliver. Rev. 60:1266; Meng et al. (2010) J. Am. Chem. Soc. 132:12690; Meng et al. (2010) ACS Nano 4:4539; Patel et al. (2008) J. Am. Chem. Soc. 130:2382; Lu et al. (2007) Small 3:1341; Shabir et al. (2011) Silicon-Neth. 3:173; Wang et al. (2010) Mol. Pharmaceut. 7:2232; Kashanian et al. (2010) Acta Biomater. 6:3566; Canham et al. U.S. Patent Publication No. 2015/0352211; Jiang et al. (2009) Phys. Status Solidi. A 206:1361; Fan et al. (2009) Phys. Status Solidi. A 206:1322; Salonen et al. (2008) J. Pharm. Sci. US 97:632; Sailor et al. (2012) Adv. Mater. 24:3779; Ruoslahti et al. (2010) J. Cell. Biol. 188:759.

The mechanism of degradation of porous silicon (pSi) is understood to involve oxidation of the silicon core to form silicon oxide, followed by hydrolysis of the resulting oxide phase to water-soluble orthosilicic acid (Si(OH)₄) or its congeners. Sailor et al. (2012) Adv. Mater. 24:3779. To prevent rapid degradation of pSi nanoparticles, various “core-shell” types of structures have been synthesized, where an inner core of pSi is surrounded by a layer or shell of more stable silicon oxide (Joo et al. (2014) Adv. Funct. Mater. 24:5688; Ray et al. (2009) J. Appl. Phys. 105:074301), titanium oxide (Betty et al. (2011) Prog. Photovoltaics 19:266; Li et al. (2014) Biosens. Bioelectron. 55:372; Jeong et al. (2014) ACS Nano 8:2977), carbon (Tsang et al. (2012) ACS Nano 6:10546; Zhou et al. (2000) Chem. Phys. Lett. 332:215; Gao et al. (2009) Phys. Chem. Chem. Phys. 11:11101), or other kinetically stable substances (Buriak (2002) Chem. Rev. 102:1271). Core-shell structures are attractive platforms for slow releasing drug delivery formulations because the synthesis of the shell can be performed in concert with drug loading in order to more effectively trap the therapeutic in the nanostructure. Fry et al. (2014) Chem. Mater. 26:2758. Furthermore, the ability of core-shell structures to enhance intensity and persistence of photoluminescence from the luminescent silicon domains in pSi has been demonstrated (Joo et al. (2014) Adv. Funct. Mater. 24:5688), which adds imaging and self-reporting drug delivery features to the nanomaterial.

Disclosed in this example is a single-step procedure to simultaneously load and protect high concentrations of siRNA in pSi nanoparticles (pSiNPs) by precipitating a shell of calcium silicate simultaneous with drug loading. Although not intended to be limiting of the invention, the source of silicate in the shell is understood to derive from local dissolution of the pSi matrix, and in solutions containing high concentrations of calcium (II) ion, it was found that Ca₂SiO₄ formation occurs primarily at the nanoparticle surface and is self-limiting. If the calcium ion solution also contains siRNA, the oligocucleotide becomes trapped in the porous nanostructure during shell formation. Again, without intending to be limiting of the invention, the insoluble calcium silicate shell is understood to slow the degradation of the porous silicon core and the release of siRNA. The porous Si core displays intrinsic photoluminescence due to quantum confinement effects, and it was found that the shell formation process leads to an increase in the external quantum yield from 0.1 to 21%, presumably due to the electronically passivating nature of the silicate shell. To demonstrate the potential for gene delivery with this system, the calcium silicate-coated pSiNPs (Ca-pSiNPs) were modified via silanol chemistry to conjugate two functional peptides, one for neuronal targeting and the other for cell penetration. The resulting construct shows significantly improved gene silencing efficacy in vitro, and it can be delivered to targeted tissues in vivo.

As illustrated in FIG. 1, mild oxidation (in aqueous media) of porous Si particles generates a thin oxide layer on the Si core. As it forms, the oxide layer becomes hydrated and solubilized, releasing Si(OH)₄ into solution. High concentrations of Ca²⁺ and siRNA present in the aqueous solution diffuse into the pores, where the Ca²⁺ ions react with the locally high concentration of Si(OH)₄, forming a precipitate that traps the siRNA payload within the nanostructure.

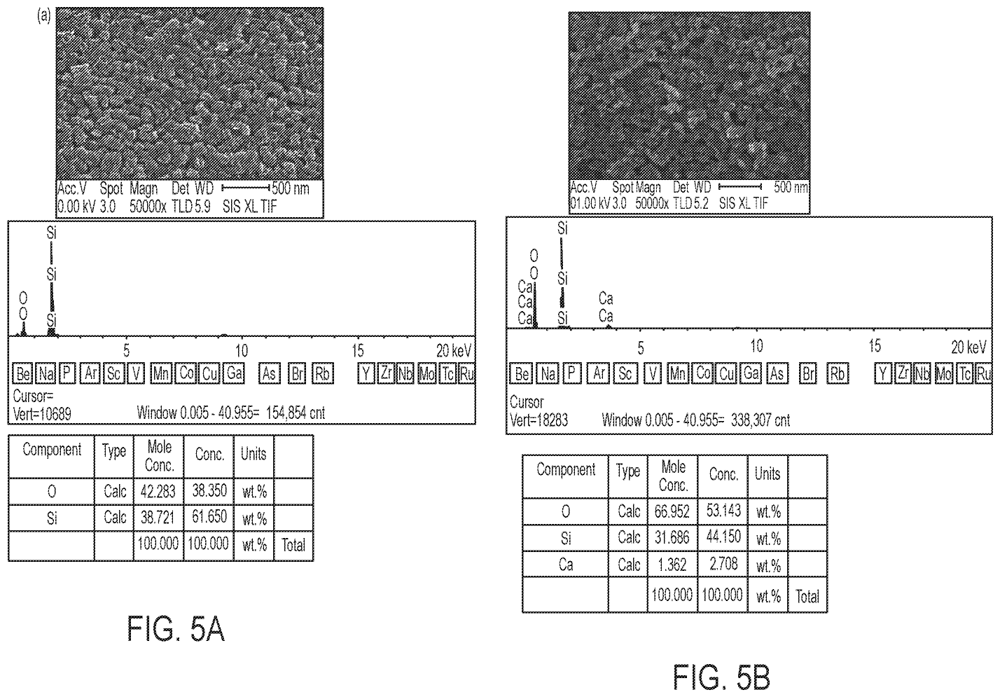

The pSiNPs of average size 180±20 nm (by dynamic light scattering) were prepared as described previously. Qin et al. (2014) Part. Part. Syst. Char. 31:252. The siRNA payload was loaded and sealed into the porous nanostructure in one step, by stirring in an aqueous solution containing the oligonucleotide and a high concentration (3 M or 4 M) of CaCl₂. The presence of silicon, calcium, and oxygen in the resulting siRNA-loaded, calcium silicate-capped pSiNPs (Ca-pSiNP-siRNA) was confirmed by energy dispersive x-ray (EDX) analysis (FIGS. 5A and 5B). No residual chloride was detected. The quantity of oxygen in the pSiNPs increased measurably upon reaction with the Ca²⁺ solution, demonstrating that pSiNPs are oxidized during the reaction.

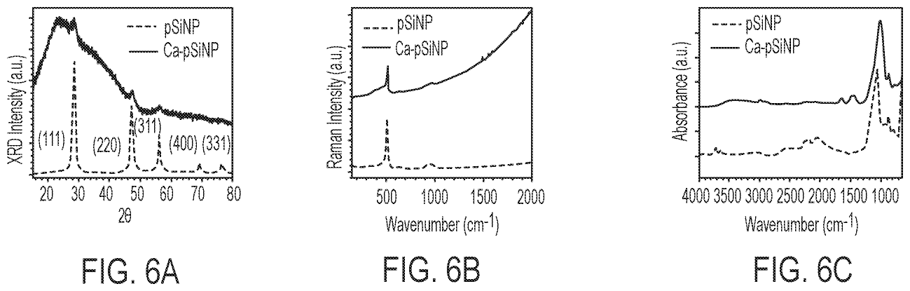

Transmission electron microscope (TEM) images (FIGS. 2A-2C) of empty pSiNP prior to calcium ion treatment, of pSiNP after treatment with Ca²⁺ (Ca-pSiNP), and of pSiNP after loading of siRNA and treatment with Ca²⁺ (Ca-pSiNP-siRNA) indicated that the reaction with Ca²⁺ generated a distinctive coating. Based on the elemental analysis and considering the low solubility of calcium silicate (Medinagonzales et al. (1988) Fert. Res. 16:3), but without intending to be bound by theory, the capping material is proposed to be dicalcium orthosilicate (Ca₂SiO₄) or a mixed phase of calcium orthosilicate, metasilicate, and silicon oxides. No crystalline calcium silicate or silicon oxide phases were observed by powder X-ray diffraction (XRD), but residual crystalline Si was observed in the XRD spectrum (FIG. 6A), the Raman spectrum (characteristic Si—Si lattice mode at 520 cm⁻¹, FIG. 6B), and the FTIR spectrum (FIG. 6C). Nitrogen adsorption-desorption isotherm analysis indicated that the total pore volume decreased by 80% (1.36±0.03 cm³/g to 0.29±0.04 cm³/g) upon conversion of pSiNP to Ca-pSiNP (FIG. 2D). Prior work showed that oxidation of pSi can result in the reduction of pore volume due to swelling of the pore walls as oxygen is incorporated into the silicon core, and this process can result in effective trapping of a payload in the pores. Sailor et al. (2012) Adv. Mater. 24:3779; Fry et al. (2014) Chem. Mater. 26:2758.

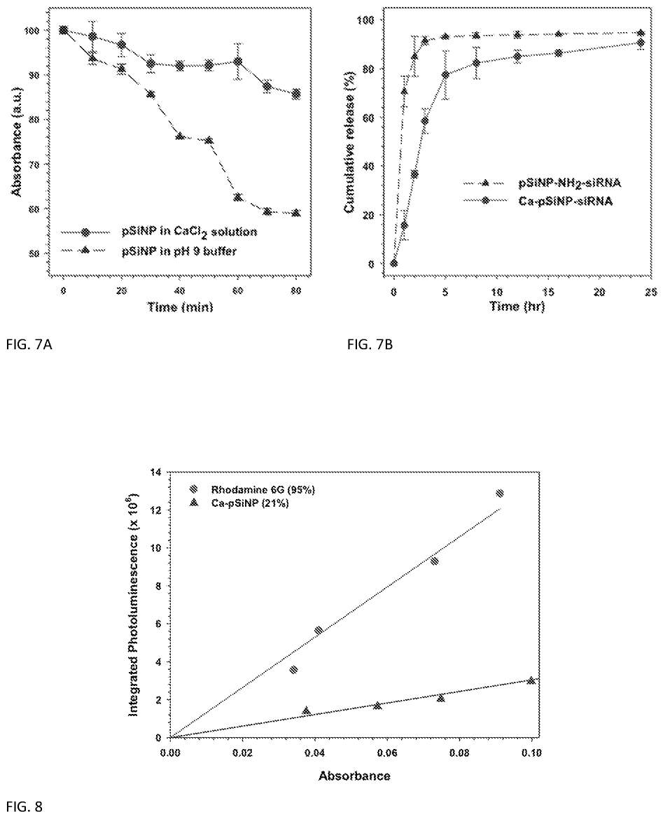

Optical absorbance measurements, used to measure the amount of elemental silicon in the solution, showed that ˜40% of the pSiNPs were degraded within 80 min in a pH 9 buffer when no calcium ion was present. However, in 3 M or 4 M CaCl₂ solution (also at pH 9), only ˜10% degradation was observed in the same time period (FIG. 7A). The calcium silicate shell also impeded release of the siRNA cargo; the Ca-pSiNP-siRNA formulation showed ˜5-fold slower release under physiologic conditions (pH 7.4 buffer, 37° C.), compared to a formulation in which siRNA was held in the pSiNPs by electrostatic means (pSiNPs modified with surface amine groups, pSiNP-NH₂, FIG. 7B). Thus the trapping reaction effectively encapsulated the siRNA payload and protected the pSi core from subsequent oxidation and hydrolysis in aqueous media.

The photoluminescence spectrum obtained at different times during the course of the reaction between pSiNPs and CaCl₂ solution showed a gradual increase in intensity (FIG. 2E). Additionally, the peak wavelength of photoluminescence blue shifted as the reaction progressed. Both of these phenomena (increase in photoluminescence intensity and blue shift of the photoluminescence spectrum) are indicative of the growth of a passivating surface layer on the silicon nanocrystallites. Joo et al. (2014) Adv. Funct. Mater. 24:5688; Petrovakoch et al. (1992) Appl. Phys. Lett. 61:943; Sa'ar (2009) J. Nanophotonics 3:032501. The observed blue shift is typical of a quantum-confined silicon nanoparticle, whose emission wavelength is strongly dependent on size and exhibits a blueshift as the quantum-confined silicon domains become smaller. Joo et al. (2015) ACS Nano 9:6233. The photoluminescence emission quantum yield (external) for the pSiNP-calcium silicate core-shell structure (Ca-pSiNP) was 21% (λₑₓ=365 nm, FIG. 8).

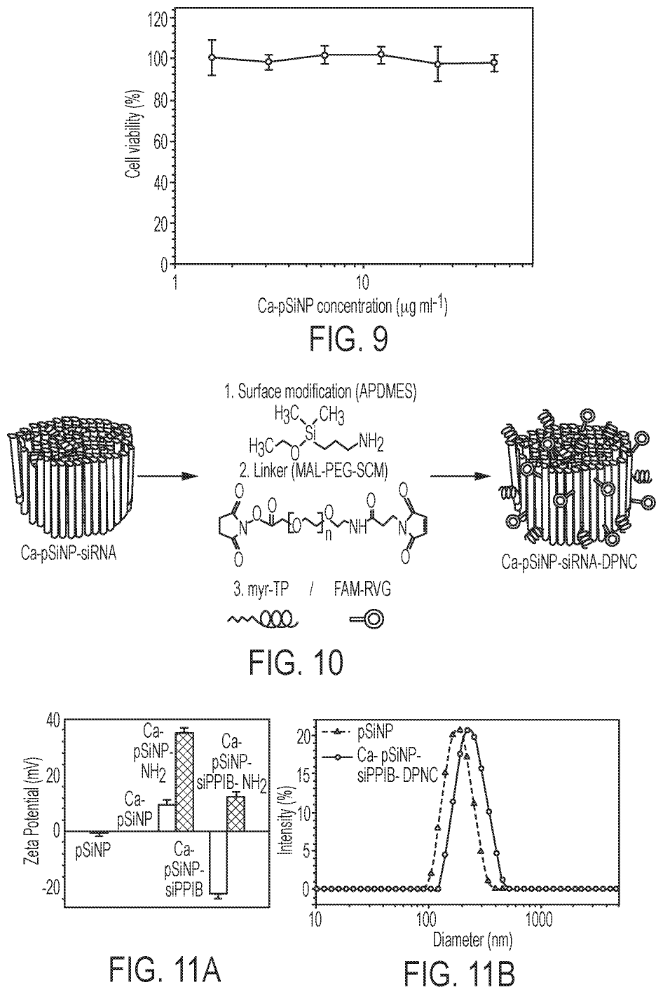

An in vitro cytotoxicity screen on cultured Neuro-2a (mouse neuroblastoma) cells showed no significant cytotoxicity of the Ca-pSiNP formulation at nanoparticle concentrations up to 50 μg/mL (FIG. 9), and so the system was loaded with a targeting and a therapeutic payload for gene silencing studies (the loading procedure is described schematically in FIG. 10). A small interfering RNA (siRNA) capable of silencing the endogenous gene (peptidylprolyl isomerase B, PPIB) was chosen to test the ability of the calcium silicate chemistry to retain, protect, and deliver a therapeutic payload for in vivo studies. The pSiNPs were loaded with siRNA against PPIB (siPPIB) in the presence of 3M CaCl₂, which resulted in ˜20 wt % siRNA content in the resulting nanoparticle (Ca-pSiNP-siRNA). The morphology of the Ca-pSiNP-siRNA construct appeared similar to the drug-free Ca-pSiNP preparation by TEM (FIG. 2C), although the surface charge (zeta potential, FIG. 11A) of Ca-pSiNP-siRNA was negative instead of positive. The positive zeta potential of the drug-free Ca-pSiNP preparation is attributed to an excess of Ca²⁺ ions at the particle surface, and the negatively charged siRNA payload neutralizes these charges to the extent that it results in an overall negative zeta potential in the Ca-pSiNP-siRNA construct.

To achieve targeted delivery and intracellular trafficking of the siRNA therapeutic, a tissue targeting peptide and a cell penetrating peptide were then grafted to the calcium silicate shell of the Ca-pSiNP-siRNA construct. A PEG linker was used to attach both of these peptides to improve systemic circulation (FIG. 10). First, the chemical coupling agent 2-aminopropyldimethylethoxysilane (APDMES) was grafted to the nanoparticle surface, generating pendant primary amine groups (Ca-pSiNP-siRNA-NH₂). Sailor et al. (2012) Adv. Mater. 24:3779. The zeta potential became more positive after the APDMES reaction for either Ca-pSi-NH₂ or Ca-pSiNP-siRNA-NH₂ formulations due to the primary amine groups on the outermost surface of the nanoparticles (FIG. 11A). Functional polyethyleneglycol (PEG) species were then grafted to Ca-pSiNP-siRNA-NH₂ via these primary amines, using a maleimide-poly(ethylene-glycol)-succinimidyl carboxy methyl ester (MAL-PEG-SCM) species. Joo et al. (2015) ACS Nano 9:6233. The succinimidyl carboxymethyl ester forms an amide bond with primary amines, and thus provides a convenient means to attach PEG to the aminated nanoparticle. The distal end of the PEG chain contained a second functional group, maleimide. Maleimide forms covalent bonds to thiols, allowing attachment of targeting and cell penetrating peptides. Two peptide species, myr-GWTLNSAGYLLGKINLKALAALAKKIL(GGCC) (SEQ ID NO:1), referred to here as “mTP,” and the rabies virus-derived peptide SFAM-(CCGG)YTIWMPENPRPGTPCDIFTNSRGKRASNG (SEQ ID NO:2), referred to as “FAM-RVG,” were prepared and conjugated to the Ca-pSiNP-siRNA-PEG formulation via reaction of the maleimide group with a cysteine thiol of the relevant peptides. Here, “SFAM” is the fluorescent label 5-carboxyfluorescein, an amine-reactive fluorophore commonly used to label biomolecules (λₑₓ/λₑₘ=495/518 nm).

Cell-penetrating peptides (CPP) such as transportan (TP) have been found to be promising auxiliaries for siRNA delivery. When CPPs are incorporated into nanoparticles, they can increase endocytic escape after internalization to increase the siRNA knockdown efficiency. However, CPPs lack cell-type specificity. To overcome this shortcoming, CPPs have been combined with cell-specific targeting peptides to generate what is known as tandem peptides, and these constructs have been shown to be very efficient siRNA delivery agents. Ren et al. (2012) ACS Nano 6:8620. In the present example, the cell-penetrating transportan peptide was attached to a myristoyl group, which contains a hydrophobic 13-carbon aliphatic chain, to enhance the hydrophobic interaction between the peptide and the lipid bilayer of the cell membrane (mTP). Ren et al. (2012) Sci. Transl. Med. 4:147ra112. The cell targeting function was accomplished with a peptide sequence from the rabies virus glycoprotein (RVG) that has demonstrated effective neuronal cell targeting efficiency in vitro and in vivo. Alvarez-Erviti et al. (2011) Nat. Biotechnol. 29:341; Lentz (1990) J. Mol. Recognit. 3:82; Kumar et al. (2007) Nature 448:39. Attachment of both RVG and mTP peptides to a Ca-pSiNP resulted in a dual peptide nanocomplex, referred to here as “Ca-pSiNP-DPNC”. Control nanoparticles containing only mTP or RVG peptides were also prepared, herein designated as Ca-pSiNP-mTP or Ca-pSiNP-RVG, respectively.

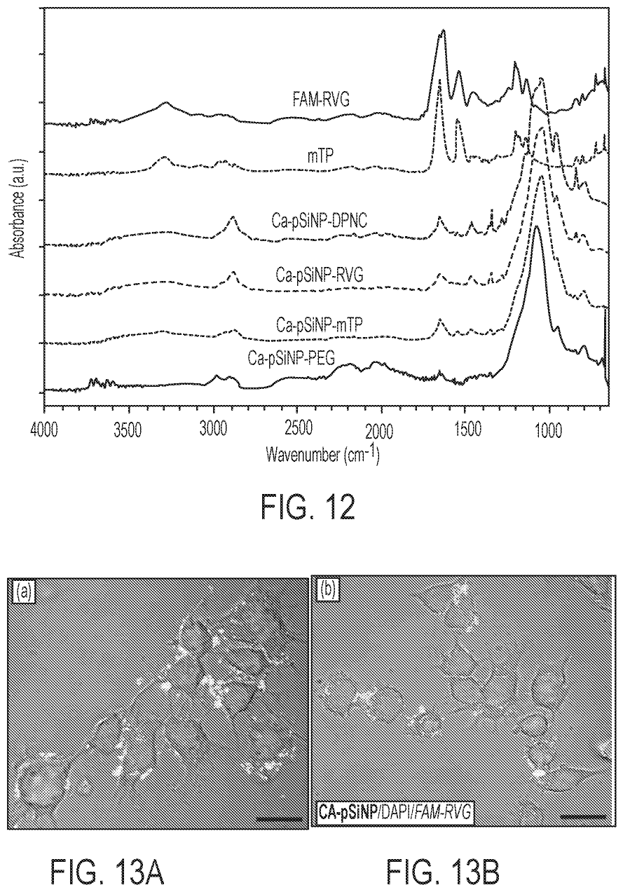

Approximately 0.086 mg of RVG was conjugated with 1 mg of Ca-pSiNP-siRNA-PEGs, determined by relative fluorescence of the FAM label. In the case of the Ca-pSiNP-siRNA-DPNC construct, approximately 0.037 mg of RVG and a comparable amount of mTP was conjugated. The Fourier transform infrared (FTIR) spectrum of Ca-pSiNP-DPNC displayed all the characteristic peaks of Ca-pSiNP-mTP and Ca-pSiNP-RVG (FIG. 12). The mean diameter of the Ca-pSiNP-siPPIB-DPNC construct was 220 nm (DLS Z-average, intensity based), representing an increase over the pSiNP starting material of 40 nm. No significant aggregates were observed in the DLS data (FIG. 11B).

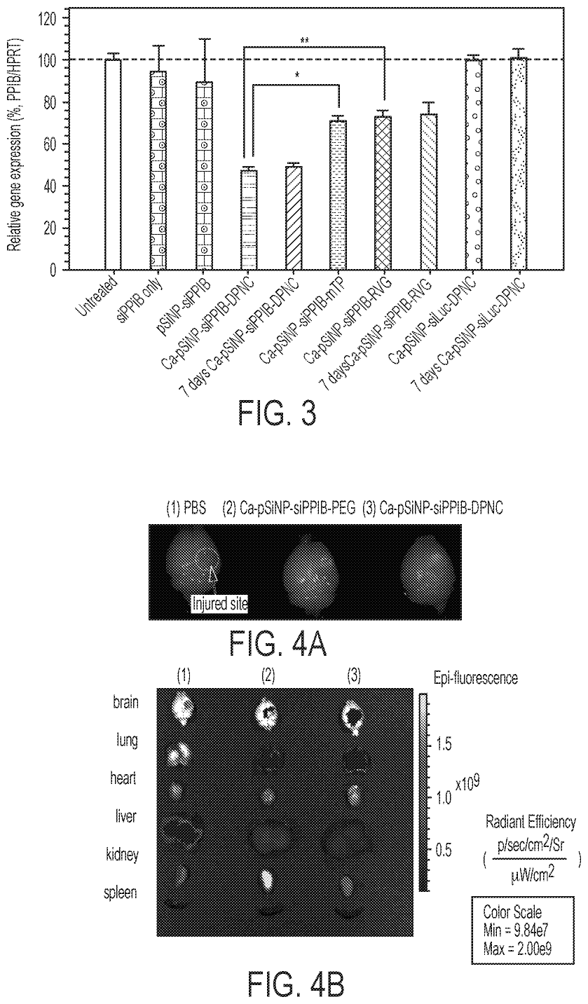

The Ca-pSiNP-siPPIB-DPNC construct effected knockdown of 52.8% of PPIB gene activity in Neuro-2a cells relative to untreated controls (FIG. 3). To eliminate the possibility that gene silencing was caused by toxicity of the nanocomplexes, a similar formulation loaded with a negative control siRNA against the luciferase gene (siLuc) was tested, and it showed no statistically significant difference relative to the untreated control. As additional controls, gene silencing efficiencies of nanoparticles containing only a cell-penetrating or only a cell-targeting peptide were tested (Ca-pSiNP-siPPIB-mTP and Ca-pSiNP-siPPIB-RVG, respectively). Both of these constructs showed some observable knockdown of PPIB gene expression (27.1-28.9% relative to untreated controls), but the silencing effect was greater with the dual peptide nanoparticle Ca-pSiNP-siPPIB-DPNC (p<0.03) compared with either peptide system individually. In the case of Ca-pSiNP-siPPIB-mTP, the gene knockdown observed in vitro is not expected to translate to in vivo activity, because the cell penetrating effect of mTP lacks cell-type specificity. On the other hand, silencing by Ca-pSiNP-siPPIB-RVG is attributed to more effective cellular localization in vitro due to specific binding of the RVG sequence to Neuro-2a cells. Additional controls using free siPPIB (not contained in a nanoparticle) and siPPIB loaded into bare pSiNPs (no Ca capping chemistry, no targeting peptides, no cell-penetrating peptides) showed no statistically significant knockdown. Furthermore, nano-constructs could be isolated and stored in ethanol for 7 days at 4° C. and still retain their PPM gene knockdown efficiency (FIG. 3).

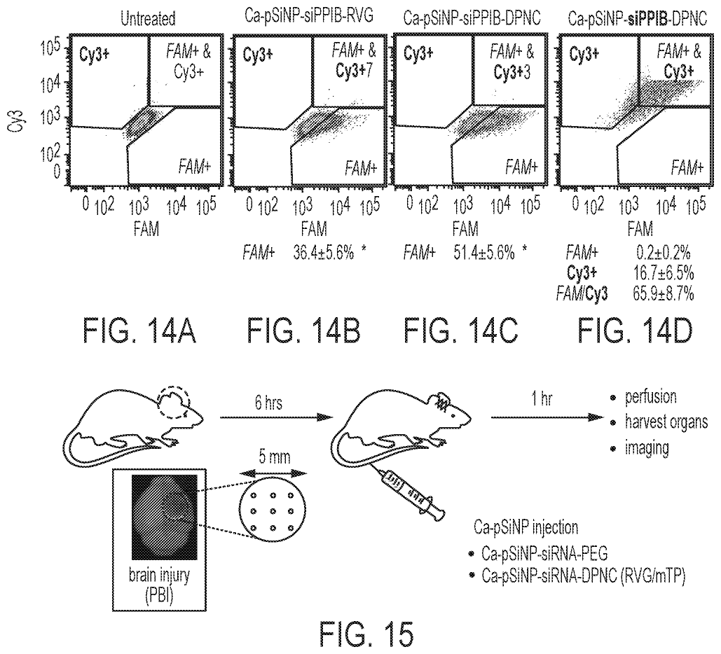

Consistent with its greater knockdown efficiency, confocal microscope images indicated that the Ca-pSiNP-siPPIB-DPNC formulation has greater affinity for Neuro-2a cells than the Ca-pSiNP-siPPIB-RVG formulation (FIGS. 13A and 13B). The Ca-pSiNP-siPPIB-DPNC formulation had approximately half the number of fluorescent FAM marker molecules on its surface compared to Ca-pSiNP-siPPIB-RVG. Even with the lower FAM fluorescence signal per particle, Neuro-2a cells treated with Ca-pSiNP-siPPIB-DPNC showed a larger FAM signal because of the greater cellular affinity of this dual peptide construct relative to the RVG-only formulation. The Ca-pSiNPs are visible in the fluorescence microscope images due to the intrinsic photoluminescence from the quantum-confined Si domains of the nanoparticle. In the case of cells treated with Ca-pSiNP-siPPIB-DPNC, the Si signal is colocalized with the signal from the FAM label on the RVG targeting peptide, and the combined signal is seen in the cytosol, indicative of cellular internalization. The cellular affinity of these two nanoparticle constructs was more accurately quantified by fluorescence-activated cell sorting (FACS) analysis (FIGS. 14A-14D), and the data show that the dual peptide nanoparticle was more efficient at targeting Neuro-2a cells than the nanoparticle that contained only the RVG peptide (51.4±5.6% vs 36.4±5.6% for Ca-pSiNP-siPPIB-DPNC and Ca-pSiNP-siPPIB-RVG, respectively (P<0.04). Separate fluorescent labels on the RVG peptide and on the siPPIB in the Ca-pSiNP-siPPIB-DPNC established that 65.9±8.7% of the cells contained both RVG and siPPIB (FIG. 14D). The results support the hypothesis that conjugating both RVG and mTP to the nanoparticle yields greater cellular affinity, which in turn generates a stronger gene knockdown effect.

As having both cell-penetrating and cell-targeting peptides on the same nanoparticle (Ca-pSiNP-siPPIB-DPNC) yielded the strongest gene knockdown in vitro, this combination was tested for in vivo gene delivery. The in vivo model involved a penetrating brain injury in mice. Significant quantities of siRNA accumulated in the site of the brain injury in the mice injected with Ca-pSiNP-siRNA-DPNC (FIG. 4). The mice (n=3) showed 2-fold greater intensity of fluorescence associated with the siRNA payload relative to the fluorescence background in saline injected control mice. There was statistically greater observed efficacy of targeting by the dual peptide Ca-pSiNP-siRNA-DPNC relative to the untargeted nanoparticles Ca-pSiNP-siRNA-PEG (p<0.02). Mice injected with the untargeted Ca-pSiNP-siRNA-PEG construct showed some siRNA fluorescence signal in the brain compared to the uninjected control mice, presumably due to passive leakage into the injury site.

In summary, this work demonstrates a self-sealing chemical procedure that can load oligonucleotides in a biodegradable and intrinsically photoluminescent nanoparticle. Substantial quantities of siRNA can be loaded (>20% by mass), and the payload is retained for therapeutically relevant timescales. The calcium silicate shell is readily modified with cell targeting (RVG peptide from rabies virus glycoprotein) and cell-penetrating (myristolated transportan) peptides, and the combination of the two peptides, along with the ability of the calcium silicate chemistry to retain and protect the siRNA payload, yields improved cellular targeting and gene knockdown in vitro. The multivalent core-shell nanoparticles circulate to deliver an siRNA payload to a brain injury in live mice, and the dual targeted nanoparticles show improved delivery of siRNA in the in vivo brain injury model relative to non-targeted nanoparticles.

### Experimental Section

Preparation of porous silicon nanoparticles: The pSiNPs were prepared following the published “perforation etching” procedure. Qin et al. (2014) Part. Part. Syst. Char. 31:252. A highly boron-doped p⁺⁺-type silicon wafer (˜1 mΩ-cm resistivity, 100 mm diameter, Virginia Semiconductor, Inc.) was anodically etched in an electrolyte composed of 3:1 (v:v) of 48% aqueous HF:ethanol. The etching waveform consisted of a square wave in which a lower current density of 46 mA cm⁻² was applied for 1.818 sec, followed by a higher current density pulse of 365 mA cm⁻² applied for 0.363 sec. This waveform was repeated for 140 cycles, generating a stratified porous silicon (pSi) film with thin, high porosity “perforations” repeating approximately every 200 nm through the porous layer. The film was removed from the silicon substrate by applying a current density of 3.4 mA cm⁻² for 250 sec in a solution containing 1:20 (v:v) of 48% aqueous HF:ethanol. The freestanding pSi film was fractured into nanoparticles of mean (Z-average, intensity based) diameter 180 nm (FIG. 11B) by immersion in deionized water and ultrasonication for ˜12 hr.

Preparation of calcium silicate-coated, siRNA-loaded porous silicon nanoparticles (Ca-pSiNP-siRNA): A stock solution 4 M in calcium chloride (CaCl₂) were prepared by adding 2.25 g of solid CaCl₂ (MW: 110.98, Anhydrous, Spectrum chemicals) to 5 mL of RNAse-free water. The solution was centrifuged to remove any precipitates and stored at 4° C. before use. For oligonucleotide loading, three kinds of duplexed siRNA constructs for the knockdown of PPIB(1), PPIB(2), and Luciferase was synthesized by Dharmacon Inc. with 3′-dTdT overhangs. Ambardekar et al. (2011) Biomaterials 32:1404; Waite et al. (2009) BMC Biotechnol. 9:38. For PPIB gene against siRNA (siPPIB), siPPIB(1) and siPPIB(2) were obtained, respectively, and used 1:1 mixture of siPPIB(1):siPPIB(2) to cover broad range of PPIB gene on the siRNA sequence sense 5′-CAA GUU CCA UCG UGU CAU C dTdT-3′ (SEQ ID NO:3) and antisense 5′-GAU GAC ACG AUG GAA CUU G dTdT-3′ (SEQ ID NO:4) for siPPIB(1) and sense 5′-GAA AGA GCA UCU AUG GUG A dTdT-3′ (SEQ ID NO:5) and antisense 5′-UCA CCA UAG AUG CUC UUU C dTdT-3′ (SEQ ID NO:6) for siPPIB(2). Luciferase gene against siRNA (siLuc) was obtained on the siRNA sequence sense 5′-CUU ACG CUG AGU ACU UCG A dTdT-3′ (SEQ ID NO:7) and antisense 5′-UCG AAG UAC UCA GCG UAA G dTdT-3′ (SEQ ID NO:8). The pSiNPs (1 mg) were dispersed in the oligonucleotide solution (150 μL, 150 μM in siRNA) and added to the CaCl₂ stock solution (850 μL). The mixture was agitated for 60 min and purified by successive dispersion in/centrifugation from RNAse free water, 70% ethanol, and 100% ethanol. To analyze siRNA loading efficiency, supernatants from each centrifugation step were collected and assayed for free siRNA using a NanoDrop 2000 spectrophotometer (Thermo Scientific, ND-2000). As a control, Ca-pSiNPs without siRNA were prepared in the same manner as described above, but excluding the added siRNA.

Conjugation of peptides to Ca-pSiNP: As-prepared Ca-pSiNP-siRNA, Ca-pSiNP or pSiNP samples (1 mg) were suspended in absolute ethanol (1 mL), an aliquot (20 μL) of aminopropyldimethylethoxysilane (APDMES) was added, and the mixture was agitated for 2 h. The aminated nanoparticles (Ca-pSiNP-siRNA-NH₂, Ca-pSiNP-NH₂, or pSiNP-NH₂) were then purified three times by centrifugation from absolute ethanol to eliminate unbound APDMES. The solutions (200 μL) of the hetero-functional linkers maleimide-PEG-succinimidyl carboxy methyl ester (MAL-PEG-SCM, MW: 5,000, Laysan Bio Inc., 5 mg/mL in ethanol) or methoxy-PEG-succinimidyl α-methylbutanoate (mPEG-SMB, Mw: 5,000, NEKTAR, 5 mg/mL in ethanol) were added to the aminated nanoparticles (1 mg in 100 μL) and agitated for 2 h. Unbound PEG linker molecules were eliminated from the PEGylated nanoparticles (Ca-pSiNP-siRNA-PEG or Ca-pSiNP-PEG) by centrifugation from ethanol three times. For the peptide-conjugated formulations, one of two peptide constructs was used: either mTP, which consists of a myristoyl group (myr) covalently attached by amide bond to the N-terminal glycine residue on the peptide sequence myr-GWTLNSAGYLLGKINLKALAALAKKIL(GGCC) (SEQ ID NO:1), or FAM-RVG, which consists of 5-carboxyfluorescein (5-FAM) attached by amide bond to the N-terminal cysteine residue on the peptide sequence 5-FAM(CCGG)YTIWMPENPRPGTPCDIFTNSRGKRASNG (SEQ ID NO:2). Both of these constructs were obtained from CPC Scientific Inc. (1 mg/mL in RNAse free water). For Ca-pSiNP-dual peptide nanocomplex (Ca-pSiNP-DPNC or Ca-pSiNP-siRNA-DPNC) synthesis, 50 μL of each peptide solution (mTP and FAM-RVG) was added to 100 μL of Ca-pSiNP-PEG in ethanol, incubated at 4° C. for 4 hours, purified three times by centrifugation, immersed in ethanol and stored at 4° C. before use. For synthesis of the single peptide conjugated Ca-pSiNP (Ca-pSiNP-siRNA-mTP or Ca-pSiNP-siRNA-RVG) control samples, 100 μL of peptide solution (mTP or FAM-RVG) was added to 100 μL of Ca-pSiNP-siRNA-PEG in ethanol, respectively. The subsequent workup was the same as described above for the Ca-pSiNP-siRNA-DPNC constructs.

Characterization: Transmission electron microscope (TEM) images were obtained with a JEOL-1200 EX II instrument. Scanning electron microscope (SEM) images and energy dispersed x-ray (EDX) data were obtained using an FEI XL30 field-emission instrument. The hydrodynamic size and zeta potential was measured by dynamic light scattering (DLS, Zetasizer ZS90, Malvern Instruments). An Ocean Optics QE-Pro spectrometer was used to obtain steady-state photoluminescence spectra (λₑₓ: 365 nm) with a 460 nm long-pass emission filter. Quantum yield measurements were performed relative to a Rhodamine 6G in ethanol standard (Q.Y. 95%). All solutions used for quantum yield measurements had optical absorbance values <0.1 at λ=365 nm. The photoluminescence intensity in the wavelength range 500-980 nm was integrated and plotted vs absorbance (FIG. 8). Nitrogen adsorption-desorption isotherms were obtained on dry particles at a temperature of 77 K with a Micromeritics ASAP 2020 instrument. Fourier transform infrared (FTIR) spectra were recorded using a Thermo Scientific Nicolet 6700 FTIR instrument. Raman spectra were obtained using a Renishaw inVia Raman microscope with 532 nm laser excitation source.

In vitro experiments: Murine Neuro-2a neuroblastoma cells (ATCC, CCL-131) were cultured in Eagle's Minimum Essential Medium (EMEM) containing 10% fetal bovine serum (FBS). Cytotoxicity of the synthesized nanoparticles was assessed using the Molecular Probes Live/Dead Viability/Cytotoxicity Kit (Molecular Probes, Invitrogen). Yee et al. (2006) Adv. Ther. 23:511. This kit uses 2 probes, Calcein AM for live cell staining (λₑₓ/λₑₘ=494/517 nm) and Ethidium homodimer-1 (EthD-1) for dead cell staining (λₑₓ/λₑₘ=528/617 nm). Neuro-2a cells (3000 cells/well) were treated with nanoparticles in triplicate in a 96-well plate. After 48 hrs, each well was washed and treated with the assay solution consisting of 4 μM EthD-1 and 2 μM Calcein AM in Dulbecco's phosphate buffered saline. After 45 min incubation at room temperature in the assay solution, well plates were read with a fluorescence plate reader (Gemini XPS spectrofluorometer, Molecular Devices, Inc.) using excitation, emission, and cutoff wavelengths 485/538/515 nm and 544/612/590 nm, respectively. A total of 15 wells per treatment group were evaluated, and plotted as a percentage of untreated control fluorescence intensity.

Neuro-2a cells treated with nanoparticles were visualized with a confocal microscope (Zeiss LSM 710 NLO), using a 40× oil immersion objective. Cells were seeded onto the coverslips (BD Biocoat Collagen Coverslip, 22 mm), incubated with nanoparticles for 2 hrs, washed three times with PBS, fixed with 4% paraformaldehyde, nucleus stained with DAPI and mounted (Thermo Fisher Scientific, Prolong Diamond Antifade Mountant with DAPI). Neuro-2a cells treated with nanoparticles were quantified to demonstrate cellular affinity and siRNA delivery efficiency by FACS analysis (LSR Fortessa).

In order to investigate knockdown efficiency in vitro, real-time quantitative reverse transcription polymerase chain reaction (RT-qPCR, Stratagene Mx3005P qPCR system) analysis was performed to examine PPIB mRNA expression. Neuro-2a cells were seeded in 24-well plates (4×10⁴ cells per well), and incubated with siRNA-loaded nanoparticles, with concentration corresponding to 100 nM of siRNA. After 48 hrs, cells were harvested and total RNA was isolated following the manufacturer's protocol (Qiagen, Valencia, Calif.). Isolated RNA was transcribed into cDNA following the manufacturer's protocol (Bio-Rad, iScript cDNA Synthesis Kit). Synthesized cDNA was subjected to qPCR analysis using SYBR Green PCR Master Mix. Primer sequences for PPIB as a target mRNA amplification and HPRT as a reference mRNA amplification are described below. PPIB forward: GGAAAGACTGTTCCAAAAACAGTG (SEQ ID NO:9), PPM reverse: GTCTTGGTGCTCTCCACCTTCCG (SEQ ID NO:10); HPRT forward: GTCAACGGGGGACATAAAAG (SEQ ID NO:11), HPRT reverse: CAACAATCAAGACATTCTTTCCA (SEQ ID NO:12). All procedures were performed in triplicate.

In vivo experiments: All animal experiments were performed under protocols approved by the MIT Institutional Animal Care and Use Committees (IACUC) and the Sanford Burnham Prebys Medical Discovery Institute Committee on Animal Use and Care. All housing and care of laboratory animals used in this study conformed to the NIH Guide for the Care and Use of Laboratory Animals in Research (see document 180F22) and all requirements and regulations issued by the USDA, including regulations implementing the Animal Welfare Act (P.L. 89-544) as amended (see document 18-F23). The in vivo model involved a penetrating brain injury in mice. First, a 5 mm diameter portion of the skull on the right hemisphere of the mouse was removed. Wounds were induced using a 21 gauge needle in a 3×3 grid for a total of 9 wounds, each 3 mm deep. After induction of injuries, the skull was replaced (FIG. 15). The mice were injected with nanoparticle constructs 6 hours post-injury via the tail vein. To quantify delivery efficiency of the siRNA cargo to the targeted injury site, Dy677-labeled (λₑₘ=700 nm) siRNA was loaded into Ca-pSiNP-PEG and Ca-pSiNP-DPNC and each of these formulations were injected into separate mice. After 1 hour of circulation the mice were perfused and the organs harvested.

Fluorescence images of harvested organs were obtained using conventional IVIS 200, xenogen, and Pearl Trilogy, Li-Cor imaging systems.

Statistical analysis: All data presented herein are expressed as the means±standard error of the mean. Significance testing was conducted using two-tailed Student's t test. Unless otherwise indicated, p<0.05 was considered statistically significant.

### Alternative Porous Silicon Metal Silicate Core-Shell Particles

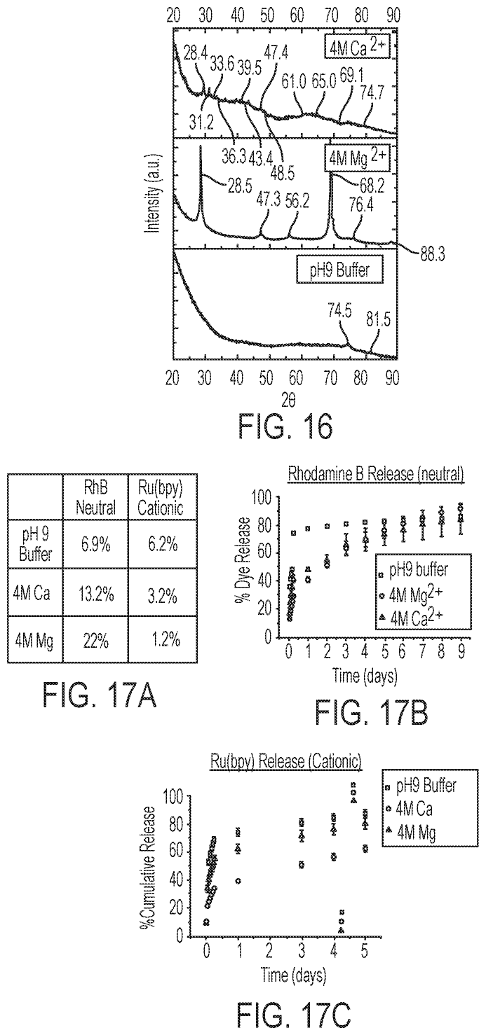

Alternative core-shell particle structures have also been prepared for further investigation and characterization. FIG. 16 shows X-ray diffraction spectra of porous silicon microparticles (pSiMPs) generated by ultrasonication of electrochemically etched porous silicon particles in solutions of 4 M calcium chloride, 4 M magnesium chloride, and pH 9 buffer. The X-ray diffraction spectrum of the pSiMPs treated with pH 9 buffer shows no significant peaks, indicating that the pSiMPs are mostly oxidized. Particles formed from magnesium chloride show little degradation or oxidation but instead display a strong spectrum for crystalline silicon. This observation indicates a likely stronger and more stable magnesium-silicon interaction due to either electrostatic adsorption of the metal or amorphous silicate formation. Particles formed from calcium chloride not only display some peaks from crystalline silicon, but also more peaks that may arise from crystalline calcium silicate bonding. By comparison with the particles formed from magnesium, however, the peaks are much less intense, possibly as a result of a thinner or less uniform shell formation around the silicon matrix.

The pore structure of pSiMP (pH 9 buffer), Mg-pSiMP, and Ca-pSiMP are characterized by nitrogen adsorption-desorption isotherm analysis (Table 1). Although no further oxidation of crystalline silicon can occur for the thermally oxidized pSiMPs, formation of calcium and magnesium layers within the pores was observed by significant reduction of the pore volume.

TABLE 1
BET and BJH calculation of surface area, average pore volume, and
average pore diameter from nitrogen porosimetry measurements.

| | | pH9 Buffer | Mg | Ca |
|---|---|---|---|---|
| | Pristine | Treatment | Treatment | Treatment |
| BET Surface Area | 210.1 m²/g | 211.8 m²/g | 136.9 m²/g | 122.7 m²/g |
| BJH Adsorption | 0.709 cm³/g | 0.822 cm³/g | 0.416 cm³/g | 0.460 cm³/g |
| Cumulative Pore | | | | |
| Volume | | | | |
| BJH Adsorption | 15.9 nm | 19.99 nm | 13.8 nm | 17.2 nm |
| Average Pore | | | | |
| Diameter | | | | |

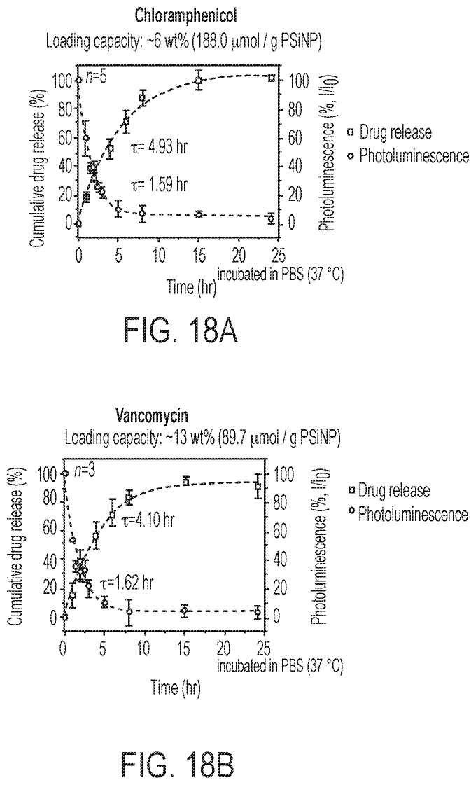

As mentioned above, anionic molecules including siRNA, microRNA, and calcein can be loaded on porous silicon particles at over 20 wt % loading efficiency during calcium silicate formation due to favorable electrostatic interactions. Loading and release of cationic or zwitterionic molecules, such as Ru(bpy), chloramphenicol, vancomycin, and rhodamin B, on porous silicon particles have also been assessed. In particular, the loading efficiency of zwitterionic (rhodamine B) or cationic (Ru(bpy)) molecules is lower than anionic molecules but displays longer sustained release due to the trapping mechanism (FIGS. 17A-17C). The loading efficiency, release kinetics, and photoluminescence profile of Ca-pSiNPs loaded with the antibiotics chloramphenicol and vancomycin are shown in FIGS. 18A-18B. The incorporated drug molecules were gradually released at physiological condition, correlating with photoluminescence decrease profile, but the release kinetics was somewhat slower than the photoluminescence decrease profile.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

What is claimed is:

1. A composition for delivering a therapeutic agent comprising:
a particle comprising a porous silicon core;
a layer on the surface of the porous silicon core comprising a metal silicate; and
a therapeutic agent.

2. The composition of claim 1, wherein the layer on the surface of the particle is formed by treating a porous silicon precursor particle with an aqueous solution comprising the therapeutic agent and a metal salt.

3. The composition of claim 2, wherein the aqueous solution comprises a concentration of metal salt of at least 0.1 molar.

4. The composition of claim 1, wherein the layer on the surface of the particle comprises a divalent metal silicate.

5. The composition of claim 4, wherein the layer on the surface of the particle comprises a calcium silicate.

6. The composition of claim 1, wherein the porous silicon core has a diameter of about 1 nm to about 1 cm.

7. The composition of claim 6, wherein the layer on the surface of the porous silicon core has a thickness of between 1 and 90 percent of the diameter of the core.

8. The composition of claim 1, wherein the particle is a photoluminescent particle.

9. The composition of claim 8, wherein the particle emits light in a range from 500 nm to 1000 nm.

10. The composition of claim 1, wherein the porous silicon core comprises an etched crystalline silicon material.

11. The composition of claim 10, wherein the porous silicon core comprises an electrochemically etched crystalline silicon material.

12. The composition of claim 10, wherein the porous silicon core comprises a chemical stain etched crystalline silicon material.

13. The composition of claim 1, wherein the porous silicon core comprises a microporous etched silicon material.

14. The composition of claim 13, wherein the microporous etched silicon material comprises a plurality of pores with an average pore diameter of at most about 1 nm.

15. The composition of claim 1, wherein the porous silicon core comprises a mesoporous etched silicon material.

16. The composition of claim 15, wherein the mesoporous etched silicon material comprises a plurality of pores with an average pore diameter of from about 1 nm to about 50 nm.

17. The composition of claim 1, wherein the porous silicon core comprises a macroporous etched silicon material.

18. The composition of claim 17, wherein the macroporous etched silicon material comprises a plurality of pores with an average pore diameter of from about 50 nm to about 1000 nm.

19. The composition of claim 1, wherein the therapeutic agent is a small-molecule agent, a vitamin, an imaging agent, a protein, a peptide, a nucleic acid, an oligonucleotide, an aptamer, or a mixture thereof.

20. The composition of claim 19, wherein the therapeutic agent is a negatively-charged therapeutic agent.

21. The composition of claim 20, wherein the therapeutic agent is an oligonucleotide.

22. The composition of claim 21, wherein the oligonucleotide is a DNA, an RNA, an siRNA, or a micro-RNA.

23. The composition of claim 22, wherein the oligonucleotide is an RNA.

24. The composition of claim 23, wherein the RNA is an siRNA.

25. The composition of claim 1, wherein the particle comprises a targeting agent.

26. The composition of claim 25, wherein the targeting agent is a neuronal targeting agent.

27. The composition of claim 1, wherein the particle comprises a cell-penetrating agent.

28. The composition of claim 27, wherein the cell-penetrating agent is a lipidated peptide.

29. The composition of claim 1, wherein the particle comprises a targeting agent and a cell-penetrating agent.

30. The composition of claim 1, wherein the porous silicon core comprises an oxidized porous silicon material.

31. The composition of claim 30, wherein the oxidized porous silicon material has been oxidized at a temperature above 150° C.

32. The composition of claim 30, wherein the oxidized porous silicon material has been oxidized in air.

33. The composition of claim 30, wherein the oxidized porous silicon material has been oxidized in solution by reaction with a chemical oxidant.

34. The composition of claim 33, wherein the chemical oxidant is water, borate, tris(hydroxymethyl)aminomethane, dimethyl sulfoxide, or nitrate.

35. A pharmaceutical composition comprising the composition of any one of claims 1-34 and a pharmaceutically acceptable carrier.

36. A method of preparing a particle for delivery of a therapeutic agent comprising the steps of:
providing a porous silicon precursor particle;
treating the porous silicon precursor particle with an aqueous solution comprising the therapeutic agent and a metal salt.

37. The method of claim 36, wherein the aqueous solution comprises a concentration of metal salt of at least 0.1 molar.

38. The method of claim 36, wherein the metal salt is a divalent metal salt.

39. The method of claim 38, wherein the metal salt is a calcium salt.

40. The method of claim 36, wherein the porous silicon precursor particle has a diameter of about 1 nm to about 1 cm.

41. The method of claim 40, wherein the particle formed from the treatment comprises a layer of metal salt on the surface of the porous silicon precursor particle having a thickness of between 1 and 90 percent of the diameter of the precursor particle.

42. The method of claim 36, wherein the particle formed from the treatment is a photoluminescent particle.

43. The method of claim 42, wherein the particle formed from the treatment emits light in a range from 500 nm to 1000 nm.

44. The method of claim 36, wherein the porous silicon precursor particle comprises an etched crystalline silicon material.

45. The method of claim 44, wherein the porous silicon precursor particle comprises an electrochemically etched crystalline silicon material.

46. The method of claim 44, wherein the porous silicon precursor particle comprises a chemical stain etched crystalline silicon material.

47. The method of claim 36, wherein the porous silicon precursor particle comprises a microporous etched silicon material.

48. The method of claim 47, wherein the microporous etched silicon material comprises a plurality of pores with an average pore diameter of at most about 1 nm.

49. The method of claim 36, wherein the porous silicon precursor particle comprises a mesoporous etched silicon material.

50. The method of claim 49, wherein the mesoporous etched silicon material comprises a plurality of pores with an average pore diameter of from about 1 nm to about 50 nm.

51. The method of claim 36, wherein the porous silicon precursor particle comprises a macroporous etched silicon material.

52. The method of claim 51, wherein the macroporous etched silicon material comprises a plurality of pores with an average pore diameter of from about 50 nm to about 1000 nm.

53. The method of claim 36, wherein the therapeutic agent is a small-molecule agent, a vitamin, an imaging agent, a protein, a peptide, a nucleic acid, an oligonucleotide, an aptamer, or a mixture thereof.

54. The method of claim 53, wherein the therapeutic agent is a negatively-charged therapeutic agent.

55. The method of claim 54, wherein the therapeutic agent is an oligonucleotide.

56. The method of claim 55, wherein the oligonucleotide is a DNA, an RNA, an siRNA, or a micro-RNA.

57. The method of claim 56, wherein the oligonucleotide is an RNA.

58. The method of claim 57, wherein the RNA is an siRNA.

59. The method of claim 36, further comprising the step of:
coupling the porous silicon particle to a targeting agent, wherein the coupling step is before or after the treating step.

60. The method of claim 59, wherein the coupling step is after the treating step.

61. The method of claim 59, wherein the targeting agent is a neuronal targeting agent.

62. The method of claim 36, further comprising the step of:
coupling the porous silicon particle to a cell-penetrating agent, wherein the coupling step is before or after the treating step.

63. The method of claim 62, wherein the coupling step is after the treating step.

64. The method of claim 62, wherein the cell-penetrating agent is a lipidated peptide.

65. The method of claim 36, further comprising the step of:
coupling the porous silicon precursor particle to a targeting agent and to a cell-penetrating agent, wherein the coupling step is before or after the treating step.

66. The method of claim 36, wherein the porous silicon precursor particle comprises an oxidized porous silicon material.

67. The method of claim 66, wherein the oxidized porous silicon material has been oxidized at a temperature above 150° C.

68. The method of claim 66, wherein the oxidized porous silicon material has been oxidized in air.

69. The method of claim 66, wherein the oxidized porous silicon material has been oxidized in solution by reaction with a chemical oxidant.

70. The method of claim 69, wherein the chemical oxidant is water, borate, tris(hydroxymethyl)aminomethane, dimethyl sulfoxide, or nitrate.

71. A method of treatment comprising administration of the composition of any one of claims 1-34 to a subject in need of treatment.

72. The method of claim 71, wherein the administration is by parenteral administration.

73. The method of claim 71, wherein the administration targets neuronal tissue.

74. The method of claim 71, further comprising the step of monitoring the subject or tissues isolated from the subject.

75. The method of claim 74, wherein the monitoring step is an optical monitoring step.
